(19) United States
(12) Patent Application Publication
(10) Pub. No.: US 2007/0104780 A1
(43) Pub. Date: May 10, 2007

(54) Formulation comprising a drug of low water solubility and method of use thereof

(76) Inventors: John M. Lipari, Racine, WI (US); Didier R. Lefebvre, Mundelein, IL (US); Tzuchi R. Ju, Vernon Hills, IL (US); Kennan C. Marsh, Lake Forest, IL (US); Geoff G.Z. Zhang, Libertyville, IL (US); Jayanthy Jayanth, Buffalo Grove, IL (US); Chetan P. Pujara, Gurnee, IL (US); Howard S. Cheskin, Glencoe, IL (US); Vitomir Vucenovic, Lorrach (DE); Ping Tong, Gurnee, IL (US)

Correspondence Address: Robert DeBerardine;D-377/AP6A-1, Abbott Laboratories, 100 Abbott Park Road, Abbott Park, IL, 60064-6008, US

(21) Appl. No.: 11/552,344

(22) Filed: Oct. 24, 2006

Related U.S. Application Data
(60) Provisional application No. 60/848,649, filed on Sep. 28, 2006.
(60) Provisional application No. 60/729,834, filed on Oct. 25, 2005.

(51) Int. Cl. A61K 31/506; A61K 31/501; A61K 31/497; A61K 31/4439; A61K 31/425; A61K 31/42; A61K 31/416; A61K 9/64

(52) U.S. Cl. 424/456; 514/252.06; 514/255.05; 514/256; 514/338; 514/273; 514/379; 514/406

(57) ABSTRACT

A pharmaceutical composition comprises a drug-carrier system having a small-molecule drug of low water solubility, e.g. N-[4-(3-amino-1H-indazol-4-yl)phenyl]-N′-(2-fluoro-5-methylphenyl)urea (ABT-869), and (+)-1-(5-tert-butyl-1-yl)-3-(1H-indazol-4-yl)-urea (ABT-102), in solution in a substantially non-aqueous carrier that comprises at least one phospholipid and a pharmaceutically acceptable solubilizing agent. The drug-carrier system, when mixed with an aqueous phase, typically forms a non-gelling, substantially non-transparent liquid dispersion. The composition is suitable for administration by a suitable route, e.g. orally, to a subject in need thereof.

This application claims priority to U.S. Provisional Application Ser. No. 60/848,649 filed Sep. 28, 2006, and U.S. Provisional Application Ser. No. 60/729,834 filed Oct. 25, 2005.

### FIELD OF THE INVENTION

The present invention relates to pharmaceutical compositions comprising a poorly water-soluble drug, more particularly a small-molecule drug of low water solubility.

### BACKGROUND OF THE INVENTION

Drugs of low water solubility, for example those classified as “practically insoluble” or “insoluble” according to United States Pharmacopeia (USP) 24 (2000), p. 10, i.e., having solubility of less than about 1 part per 10,000 parts water (less than about 100 μg/ml) are notoriously difficult to formulate for oral delivery. Among other problems, bioavailability of such drugs, when administered by the oral route, tends to be very low.

Various solutions to the challenge of low oral bioavailability have been proposed for particular poorly soluble drugs. For example, U.S. Pat. No. 5,645,856 to Lacy et al. proposes formulating a hydrophobic drug with (a) an oil, (b) a hydrophilic surfactant and (c) a lipophilic surfactant that substantially reduces an inhibitory effect of the hydrophilic surfactant on in vivo lipolysis of the oil, such lipolysis being said to be a factor promoting bioavailability of the drug. Among numerous classes of hydrophilic surfactants listed are phospholipids such as lecithins.

U.S. Pat. No. 6,267,985 to Chen & Patel is directed, inter alia, to a pharmaceutical composition comprising (a) a triglyceride, (b) a carrier comprising at least two surfactants, one of which is hydrophilic, and (c) a therapeutic agent capable of being solubilized in the triglyceride, the carrier or both. It is specified therein that the triglyceride and the surfactants must be present in amounts providing a clear aqueous dispersion when the composition is mixed with an aqueous solution under defined conditions. Among extensive separate lists of exemplary ingredients, mention is made of “glyceryl tricaprylate/caprate” as a triglyceride, and phospholipids including phosphatidyl-choline as surfactants.

U.S. Pat. No. 6,451,339 to Patel & Chen mentions disadvantages of presence of triglycerides in such compositions, and proposes otherwise similar compositions that are substantially free of triglycerides, but that likewise provide clear aqueous dispersions. U.S. Pat. No. 6,309,663 to Patel & Chen proposes pharmaceutical compositions comprising a combination of surfactants said to enhance bioabsorption of a hydrophilic therapeutic agent. Phospholipids such as phosphatidylcholine are again listed among exemplary surfactants.

U.S. Pat. No. 6,464,987 to Fanara et al. proposes a fluid pharmaceutical composition comprising an active substance, 3% to 55% by weight of phospholipid, 16% to 72% by weight of solvent, and 4% to 52% by weight of fatty acid. Compositions comprising Phosal 50 PG™ (primarily comprising phosphatidylcholine and propylene glycol), in some cases together with Phosal 53 MCT™ (primarily comprising phosphatidylcholine and medium chain triglycerides), are specifically exemplified. Such compositions are said to have the property of gelling instantaneously in presence of an aqueous phase and to allow controlled release of the active substance.

U.S. Pat. No. 5,538,737 to Leonard et al. proposes a capsule containing a water-in-oil emulsion wherein a water-soluble drug salt is dissolved in the water phase of the emulsion and wherein the oil phase comprises an oil and an emulsifying agent. Among oils mentioned are medium chain triglycerides; among emulsifying agents mentioned are phospholipids such as phosphatidylcholine. Phosal 53 MCT™, which contains phosphatidylcholine and medium chain triglycerides, is reportedly used according to various examples therein.

Phospholipids together with medium chain triglycerides have also been proposed as ingredients for formulating a drug in a water-based system for topical administration. See for example U.S. patent application Publication No. 2004/0063794 of Schwarz et al. U.S. Pat. No. 5,536,729 to Waranis & Leonard proposes an oral formulation comprising rapamycin, at a concentration of about 0.1 to about 50 mg/ml, in a carrier comprising a phospholipid solution. It is stated therein that a preferred formulation can be made using Phosal 50 PG™ as the phospholipid solution. An alternative phospholipid solution mentioned is Phosal 53 MCT™.

U.S. Pat. No. 5,559,121 to Harrison et al. proposes an oral formulation comprising rapamycin, at a concentration of about 0.1 to about 100 mg/ml, in a carrier comprising N,N-dimethylacetamide and a phospholipid solution. Examples of the more preferred embodiments are shown to be prepared using Phosal 50 PG™. An alternative phospholipid solution mentioned is Phosal 53 MCT™.

Rapamycin is a high molecular weight (914.2 g/mol) compound and as such presents challenges that are qualitatively and/or quantitatively different from those presented by small-molecule drugs having lower molecular weight.

A specific illustrative small-molecule drug of low water solubility is the compound N-[4-(3-amino-1H-indazol-4-yl)phenyl]-N′-(2-fluoro-5-methylphenyl)urea (ABT-869), a multi-targeted protein tyrosine kinase (PTK) inhibitor. This compound, which has a molecular weight of 375.4 g/mol, is disclosed in International Patent Publication No. WO 2004/113304 of Abbott Laboratories, e.g., at Example 5 thereof, wherein the compound is prepared as the trifluoroacetate salt. It is stated therein that the subject compounds can be administered in the form of liposome delivery systems including multilamellar vesicles, and that liposomes can be formed from a variety of phospholipids, such as phosphatidylcholines.

Another illustrative example of small-molecule drug with low water solubility is the compound (+)-1-(5-tert-butyl-1-yl)-3-(1H-indazol-4-yl)-urea) (ABT-102), a first-in-class TRPV1 antagonist, intended for the treatment of pain. ABT-102 has a molecular weight of 348.44 g/mol and is disclosed in U.S. Pat. No. 7,015,233.

There remains a need in the pharmaceutical art for a novel liquid formulation of a small-molecule drug of low water solubility such as ABT-869 and ABT-102 that is suitable for oral administration. More particularly and without limitation, there is a need for such a formulation having at least one of the following features, advantages or benefits: acceptably high concentration of the drug (for example at least about 50 mg/ml); and acceptable bioavailability (for example at least about 20%) when administered orally.

### SUMMARY OF THE INVENTION

There is now provided a pharmaceutical composition comprising a drug-carrier system having a small-molecule drug of low water solubility in solution in a substantially non-aqueous carrier that comprises (a) at least one phospholipid and (b) a pharmaceutically acceptable solubilizing agent. The drug-carrier system, when mixed with an aqueous phase, forms a non-gelling, substantially non-transparent liquid dispersion.

There is further provided a method of delivering a small-molecule drug of low water solubility to a subject, the method comprising administering, by a suitable route of administration, a composition that comprises a drug-carrier system having the drug in solution in a substantially non-aqueous carrier comprising (a) at least one phospholipid and (b) a pharmaceutically acceptable solubilizing agent; wherein the drug-carrier system, when mixed with an aqueous phase, forms a non-gelling, substantially non-transparent liquid dispersion.

The small-molecule drug of low water solubility can illustratively be a PTK inhibitory compound of formula (I) 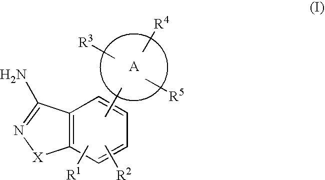
or a therapeutically acceptable salt thereof, where
A is selected from the group consisting of indolyl, phenyl, pyrazinyl, pyridazinyl, pyridinyl, pyrimidyl and thienyl;
X is selected from the group consisting of O, S and NR⁹;
R¹ and R² are independently selected from the group consisting of hydrogen, alkoxy, alkoxyalkoxy, alkoxyalkyl, alkyl, aryl, arylalkyl, aryloxy, aryloxyalkyl, halo, haloalkoxy, haloalkyl, heterocyclyl, heterocyclylalkenyl, heterocyclylalkoxy, heterocyclylalkyl, heterocyclyloxyalkyl, hydroxy, hydroxyalkoxy, hydroxy-alkyl, (NR^{a}R^{b})alkoxy, (NR^{a}R^{b})alkenyl, (NR^{a}R^{b})alkyl, (NR^{a}R^{b})alkynyl, (NR^{a}R^{b})carbonylalkenyl and (NR^{a}R^{b})carbonylalkyl;
R³, R⁴ and R⁵ are each independently selected from the group consisting of hydrogen, alkoxy, alkoxyalkoxy, alkyl, halo, haloalkoxy, haloalkyl, hydroxy and LR⁶, provided at least two of R³, R⁴ and R⁵ are other than LR⁶;
L is selected from the group consisting of (CH₂)ₘN(R⁷)C(O)N(R⁸)(CH₂)ₙ and CH₂C(O)NR⁷, where m and n are independently 0 or 1, and wherein each group is drawn with its left end attached to A;
R⁶ is selected from the group consisting of hydrogen, aryl, cycloalkyl, heterocyclyl and 1,3-benzodioxolyl, wherein the 1,3-benzodioxolyl is optionally substituted with one, two or three substituents independently selected from the group consisting of alkenyl, alkoxy, alkoxyalkyl, alkoxycarbonyl, alkyl, alkylcarbonyl, aryl, arylalkoxy, arylalkyl, aryloxy, carboxy, cyano, cycloalkyl, halo, haloalkoxy, haloalkyl, a second heterocyclyl group, heterocyclylalkyl, hydroxy, hydroxyalkyl, nitro, —NR^{c}R^{d} and (NR^{c}R^{d})alkyl;
R⁷ and R⁸ are independently selected from the group consisting of hydrogen and alkyl;
R⁹ is selected from the group consisting of hydrogen, alkenyl, alkoxyalkyl, alkyl, alkylcarbonyl, aryl, heterocyclylalkyl, hydroxyalkyl and (NR^{a}R^{b})alkyl;
R^{a} and R^{b} are independently selected from the group consisting of hydrogen, alkenyl, alkyl, alkylcarbonyl, alkylsulfonyl, aryl, arylalkyl, arylcarbonyl, arylsulfonyl, haloalkylsulfonyl, cycloalkyl, heterocyclyl, heterocyclylalkyl and heterocyclyl-sulfonyl; and
R^{c} and R^{d} are independently selected from the group consisting of hydrogen, alkyl, alkylcarbonyl, aryl, arylalkyl, cycloalkyl and heterocyclyl.
   An illustrative example of a compound of formula (I) is N-[4-(3-amino-1H-indazol-4-yl)phenyl]-N′-(2-fluoro-5-methylphenyl)urea (ABT-869).

Another small-molecule drug of low water solubility can be a TRPV1 antagonist compound of formula (III) 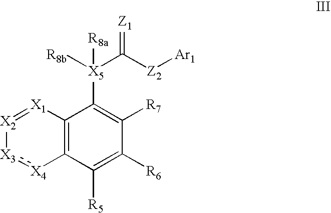
or a pharmaceutically acceptable salt or prodrug thereof, wherein

is absent or a single bond;

X₁ is N or CR₁;

X₂ is N or CR₂;

X₃ is N, NR₃, or CR₃;

X₄ is a bond, N, or CR₄;

X₅ is N or C;

provided that at least one of X₁, X₂, X₃, and X₄ is N;

Z₁ is O, NH, or S;

Z₂ is a bond, NH, or O;

Ar₁ is dihydro-1H-indenyl, 1H-indenyl, tetrahydronaphthalenyl, or dihydronaphthalenyl, wherein the Ar₁ group is optionally substituted with 1, 2, 3, 4, or 5 substituents independently selected from alkenyl, alkoxy, alkoxyalkoxy, alkoxyalkyl, alkoxycarbonyl, alkoxycarbonylalkyl, alkyl, alkylcarbonyl, alkylcarbonylalkyl, alkylcarbonyloxy, alkylthio, alkynyl, carboxy, carboxyalkyl, cyano, cyanoalkyl, formyl, formylalkyl, haloalkoxy, haloalkyl, haloalkylthio, halogen, hydroxy, hydroxyalkyl, mercapto, mercaptoalkyl, nitro, (CF₃)₂(HO)C—, —NR_{A}S(O)₂R_{B}, —S(O)₂OR_{A}, —S(O)₂R_{B}, —NZ_{A}Z_{B}, (NZ_{A}Z_{B})alkyl, (NZ_{A}Z_{B})carbonyl, (NZ_{A}Z_{B})carbonylalkyl, or (NZ_{A}Z_{B})sulfonyl, wherein Z_{A} and Z_{B} are each independently hydrogen, alkyl, alkylcarbonyl, formyl, aryl, or arylalkyl; R₁, R₃, R₅, R₆, and R₇ are each independently hydrogen, alkenyl, alkoxy, alkoxyalkoxy, alkoxyalkyl, alkoxycarbonyl, alkoxycarbonylalkyl, alkyl, alkylcarbonyl, alkylcarbonylalkyl, alkylcarbonyloxy, alkylthio, alkynyl, carboxy, carboxyalkyl, cyano, cyanoalkyl, cycloalkyl, cycloalkylalkyl, formyl, formylalkyl, haloalkoxy, haloalkyl, haloalkylthio, halogen, hydroxy, hydroxyalkyl, mercapto, mercaptoalkyl, nitro, (CF₃)₂(HO)C—, —NR_{A}S(O)₂R_{B}, —S(O)₂OR_{A}, —S(O)₂R_{B}, —NZ_{A}Z_{B}, (NZ_{A}Z_{B})alkyl, (NZ_{A}Z_{B})carbonyl, (NZ_{A}Z_{B})carbonylalkyl or (NZ_{A}Z_{B})sulfonyl;

R₂ and R₄ are each independently hydrogen, alkenyl, alkoxy, alkoxyalkoxy, alkoxyalkyl, alkoxycarbonyl, alkoxycarbonylalkyl, alkyl, alkylcarbonyl, alkylcarbonylalkyl, alkylcarbonyloxy, alkylthio, alkynyl, carboxy, carboxyalkyl, cyano, cyanoalkyl, cycloalkyl, cycloalkylalkyl, formyl, formylalkyl, haloalkoxy, haloalkyl, haloalkylthio, halogen, hydroxy, hydroxyalkyl, mercapto, mercaptoalkyl, nitro, (CF₃)₂(HO)C—, —NR_{A}S(O)₂R_{B}, —S(O)₂OR_{A}, —S(O)₂R_{B}, —NZ_{A}Z_{B}, (NZ_{A}Z_{B})alkyl, (NZ_{A}Z_{B})alkylcarbonyl, (NZ_{A}Z_{B})carbonyl, (NZ_{A}Z_{B})carbonylalkyl, (NZ_{A}Z_{B})sulfonyl, (NZ_{A}Z_{B})C(═NH)—, (NZ_{A}Z_{B})C(═NCN)NH—, or (NZ_{A}Z_{B})C(═NH)NH—;

R_{A} is hydrogen or alkyl;

R_{B} is alkyl, aryl, or arylalkyl;

R₈ₐ is hydrogen or alkyl; and

R_{8b} is absent, hydrogen, alkoxy, alkoxycarbonylalkyl, alkyl, alkylcarbonyloxy, alkylsulfonyloxy, halogen, or hydroxy;

provided that R_{8b} is absent when X₅ is N.

An example of a compound of formula (III) is (+)-1-(5-tert-butyl-1-yl)-3-(1H-indazol-4-yl)-urea) (ABT-102),

There is still further provided a pharmaceutical composition comprising a drug-carrier system having a compound of formula (I), e.g. ABT-869, in solution in a substantially non-aqueous carrier that comprises (a) at least one phospholipid and (b) a pharmaceutically acceptable solubilizing agent.

There is still further provided a pharmaceutical composition comprising a drug-carrier system having a compound of formula (III), e.g., ABT-102, in solution in a substantially non-aqueous carrier that comprises (a) at least one phospholipid and (b) a pharmaceutically acceptable solubilizing agent.

There is still further provided a method of delivering a compound of formula (I), e.g. ABT-869, to a subject, the method comprising administering, by a suitable route of administration, a composition that comprises a drug-carrier system having the drug in solution in a substantially non-aqueous carrier comprising (a) at least one phospholipid and (b) a pharmaceutically acceptable solubilizing agent.

There is still further provided a method of delivering a compound of formula (III), e.g. ABT-102, to a subject, the method comprising administering, by a suitable route of administration, a composition that comprises a drug-carrier system having the drug in solution in a substantially non-aqueous carrier comprising (a) at least one phospholipid and (b) a pharmaceutically acceptable solubilizing agent.

There is still further provided a method of treating a condition in a subject for which a PTK inhibitor is indicated, the method comprising administering to the subject, by a suitable route of administration, a composition that comprises a liquid drug-carrier system having a compound of formula (I), e.g. ABT-869, in solution in a substantially non-aqueous liquid carrier comprising (a) at least one phospholipid and (b) a pharmaceutically acceptable solubilizing agent.

There is still further provided a method of treating a condition in a subject for which a TRPV1 antagonist is indicated, the method comprising administering to the subject, by a suitable route of administration, a composition that comprises a liquid drug-carrier system having a compound of formula (III), e.g. ABT-102, in solution in a substantially non-aqueous liquid carrier comprising (a) at least one phospholipid and (b) a pharmaceutically acceptable solubilizing agent.

According to any of the above methods, a preferred route of administration is the oral route.

### DETAILED DESCRIPTION

A “drug-carrier system” herein comprises a carrier having a drug homogeneously distributed therein. In compositions of the present invention the drug is in solution in the carrier, and, in some embodiments, the drug-carrier system constitutes essentially the entire composition. In other embodiments, the drug-carrier system is encapsulated within a capsule shell that is suitable for oral administration; in such embodiments the composition comprises the drug-carrier system and the capsule shell.

The carrier and the drug-carrier system are typically liquid, but in some embodiments the carrier and/or the drug-carrier system can be solid or semi-solid. For example, the drug-carrier system can comprise a solid solution of the drug in the carrier, as can illustratively be prepared by dissolving the drug in a carrier at a temperature above the melting or flow point of the carrier, and cooling the resulting solution to a temperature below the melting or flow point to provide the solid solution. Alternatively or in addition, the carrier can comprise a solid substrate wherein or whereon a solution of the drug as described herein is adsorbed.

A composition of the invention can be useful for delivery of the drug to a subject in need thereof by any suitable route of administration, including without limitation parenteral, oral, sublingual, buccal, intranasal, pulmonary, topical, transdermal, intradermal, ocular, otic, rectal, vaginal, intragastric, intrasynovial and intra-articular routes. In a presently preferred embodiment, the composition is adapted for oral administration.

The terms “oral administration” and “orally administered” herein refer to administration to a subject per os, that is, administration wherein the composition is immediately swallowed. “Oral administration” is distinguished herein from intraoral administration, e.g. sublingual or buccal administration or topical administration to intraoral tissues such as periodontal tissues, that does not involve immediate swallowing of the composition.

Drugs useful herein are small-molecule compounds, i.e., compounds having a molecular weight, excluding counterions in the case of salts, not greater than about 750 g/mol, typically not greater than about 500 g/mol.

Further, drugs useful herein are compounds of low solubility in water, for example having solubility of less than about 100 μg/ml, in most cases less than about 30 μg/ml. The present invention can be especially advantageous for drugs that are essentially insoluble in water, i.e., having a solubility of less than about 10 μg/ml. It will be recognized that aqueous solubility of many drugs is pH dependent; in the case of such drugs the solubility of interest herein is at a physiologically relevant pH, for example a pH of about 1 to about 8. Thus, in various embodiments, the drug has a solubility in water, at least at one point in a pH range from about 1 to about 8, of less than about 100 μg/ml, for example less than about 30 μg/ml, or less than about 10 μg/ml. For example, ABT-869 has a solubility in water at pH 1 of only about 1.7 g/ml, and at pH 5 even lower—about 27 ng/ml; ABT-102 has a solubility in water at pH 1.1 of only about 102 ng/ml, and at pH 6.8 of about 57.3 ng/ml.

The drug can address any biochemical target and have any therapeutic utility, except that the target should be one accessible via systemic delivery, for example oral delivery, of the drug. Non-limiting illustrative examples of suitable drugs include ABT-869, ABT-102, acetohexamide, alprazolam, benzthiazide, carboquone, celecoxib, chlorambucil, cilostazol, dexamethasone, digoxin, estradiol, etodolac, exemestane, fenofibrate, fenticonazole, finasteride, furosemide, griseofulvin, haloperidol, hydrochlorothiazide, hydrocodone, indomethacin, isotretinoin, lansoprazole, latanoprost, letrozole, lopinavir, loratadine, lorazepam, megestrol acetate, mestranol, methylprednisolone, mofezolac, nabumetone, nitrazepam, olanzapine, oxazepam, paricalcitol, progesterone, pyrimethamine, rofecoxib, salsalate, simvastatin, spironolactone, sulfabenzamide, sulindac, tetrahydrocannabinol, thalidomide, tretinoin, valdecoxib, etc., and combinations of such drugs.

In one embodiment, the drug is a PTK inhibitory compound, for example a compound of formula (I) above. More particularly, the drug can be a compound of formula (II) 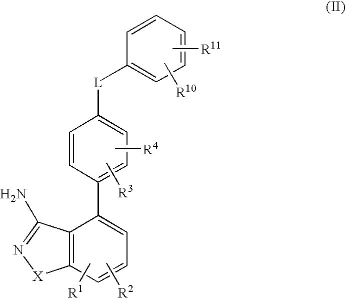
or a therapeutically acceptable salt thereof, where
X is selected from the group consisting of O, S and NR⁹;
R¹ and R² are independently selected from the group consisting of hydrogen, alkoxy, alkoxyalkoxy, alkoxyalkyl, alkyl, aryloxy, aryloxyalkyl, halo, haloalkoxy, haloalkyl, heterocyclyl, heterocyclylalkenyl, heterocyclylalkoxy, heterocyclyl-alkyl, heterocyclyloxyalkyl, hydroxy, hydroxy-alkoxy, hydroxyalkyl, (NR^{a}R^{b})alkoxy, (NR^{a}R^{b})alkenyl, (NR^{a}R^{b})alkyl, (NR^{a}R^{b})carbonylalkenyl and (NR^{a}R^{b})carbonylalkyl;
R³ and R⁴ are independently selected from the group consisting of hydrogen, alkoxy, alkyl, halo, haloalkoxy, haloalkyl and hydroxy;
L is selected from the group consisting of (CH₂)ₘN(R⁷)C(O)N(R⁸)(CH₂)ₙ and CH₂C(O)NR⁷, where m and n are independently 0 or 1, and wherein each group is drawn with its left end attached to the ring substituted with R³ and R⁴;
R⁷ and R⁸ are independently selected from the group consisting of hydrogen and alkyl;
R⁹ is selected from the group consisting of hydrogen, alkenyl, alkoxyalkyl, alkyl, alkylcarbonyl, aryl, heterocyclylalkyl, hydroxyalkyl and (NR^{a}R^{b})alkyl;
   R¹⁰ and R¹¹ are independently selected from the group consisting of hydrogen, alkoxy, alkoxyalkyl, alkoxycarbonyl, alkyl, aryloxy, arylalkyl, carboxy, cyano, halo, haloalkoxy, haloalkyl, hydroxy, hydroxyalkyl, nitro and —NR^{c}R^{d};
   R^{a} and R^{b} are independently selected from the group consisting of hydrogen, alkyl, alkylcarbonyl, alkylsulfonyl, arylsulfonyl, haloalkylsulfonyl and heterocyclyl-sulfonyl; and
   R^{c} and R^{d} are independently selected from the group consisting of hydrogen, alkyl, alkylcarbonyl, aryl, arylalkyl, cycloalkyl, cycloalkylalkyl, heterocyclyl and heterocyclylalkyl.

Compounds of formulas (I) and (II), and methods of preparation of such compounds, are disclosed in above-cited International Patent Publication No. WO 2004/113304, incorporated herein by reference in its entirety. Terms for substituents used herein are defined exactly as in that publication.

Illustratively, the drug can be a compound of formula (II) wherein X is NH; R¹, R², R³ and R⁴ are each hydrogen; and L is NHC(O)NH. Such a compound is an N-[4-(3-amino-1H-indazol-4-yl)phenyl]-N′-phenylurea, optionally substituted on the N′-phenyl ring as specified by R¹⁰ and R¹¹ above.

R¹⁰ and R¹¹ in such a compound can illustratively be independently selected from the group consisting of hydrogen, alkyl and halo. Alkyl (more particularly C₁₋₃ alkyl, e.g., methyl or ethyl) and/or halo (e.g., fluoro, chloro, bromo or iodo) substitutions are illustratively at the 2- and/or 5-positions on the N′-phenyl ring, but other substitution patterns can also be useful. ABT-869 is a specific example of such a compound having 2-fluoro and 5-methyl substitution on the N′-phenyl ring.

In one embodiment the PTK inhibitory compound is multi-targeted, i.e., an inhibitor of at least two kinase classes, for example a VEGF (vascular endothelial growth factor) receptor tyrosine kinase and a PDGF (platelet-derived endothelial growth factor) receptor tyrosine kinase. ABT-869 illustratively inhibits a range of VEGF and PDGF receptor tyrosine kinases. It is believed that a multi-targeted PTK inhibitor such as ABT-869 can disrupt tumor progression in neoplastic disease by a plurality of mechanisms.

A composition as provided herein having as the drug any specific compound disclosed in above-cited International Patent Publication No. 2004/113304 is expressly contemplated as an embodiment of the present invention.

In another embodiment, the drug is a TRPV1 antagonist, for example a compound of formula (III) above. More particularly, the drug can be a compound of formula (IV) 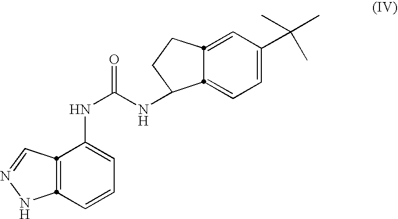
Compounds of formulas (III) and (IV) and methods of preparation of such compounds are disclosed in above-cited U.S. Pat. No. 7,015,233, incorporated herein by reference in its entirety. ABT-102 inhibits TRPV1 receptors and it is useful in treating urinary disorders, such as bladder dysfunction and urinary incontinence, as well as neuropathic pain, inflammatory pain, and migraine.

A small-molecule drug of low water solubility is present in the composition in an amount that can be therapeutically effective when the composition is administered to a subject in need thereof according to an appropriate regimen. Typically, a unit dose of the drug, which can be administered at an appropriate frequency, e.g., one to about four times a day, or in some situations less frequently than once daily, is about 0.01 to about 1,000 mg, depending on the drug in question. Illustratively, for example where the drug is ABT-869, the unit dose can be about 1 to about 500 mg, more typically about 10 to about 300 mg or about 20 to about 200 mg. Where the composition comprises a capsule shell enclosing the drug-carrier system, a unit dose can be deliverable in a single capsule or a small plurality of capsules, most typically 1 to 2 capsules.

The higher the unit dose, the more desirable it becomes to select a carrier that permits a relatively high concentration of the drug in solution therein. Typically, the concentration of drug in the drug-carrier system is at least about 10 mg/ml, e.g., about 10 to about 500 mg/ml, but lower and higher concentrations can be acceptable or achievable in specific cases. Illustratively, for example where the drug is ABT-869, the drug concentration in various embodiments is at least about 10 mg/ml, e.g., about 10 to about 400 mg/ml, or at least about 50 mg/ml, e.g. about 50 to about 300 mg/ml, or at least about 67 mg/ml, e.g. about 67 to about 250 mg/ml, or at least about 100 mg/ml, e.g., about 100 to about 200 mg/ml.

In a composition of the invention, the drug is “in solution” in the carrier. This should be taken to mean that substantially all of the drug is in solution, i.e., no substantial portion of the drug is in solid (e.g., crystalline) form, whether dispersed, for example in the form of a suspension, or not. In practical terms, this means that the drug must normally be formulated at a concentration below its limit of solubility in the carrier. It will be understood that the limit of solubility can be temperature-dependent, thus selection of a suitable concentration should take into account the range of temperatures to which the composition is likely to be exposed in normal storage, transport and use.

The carrier is “substantially non-aqueous”, i.e., having no water, or an amount of water that is small enough to be, in practical terms, essentially non-deleterious to performance or properties of the composition. Typically, the carrier comprises zero to less than about 5% by weight water. It will be understood that certain ingredients useful herein can bind small amounts of water on or within their molecules or supramolecular structures; such bound water if present does not affect the “substantially non-aqueous” character of the carrier as defined herein.

As indicated above, the carrier comprises two essential components: at least one phospholipid, and a pharmaceutically acceptable solubilizing agent for the at least one phospholipid. The solubilizing agent, or the combination of solubilizing agent and phospholipid, also solubilizes the drug, although other carrier ingredients such as a surfactant optionally present in the carrier can in some circumstances provide enhanced solubilization of the drug.

Any pharmaceutically acceptable phospholipid or mixture of phospholipids can be used. In general such phospholipids are phosphoric acid esters that yield on hydrolysis phosphoric acid, fatty acid(s), an alcohol and a nitrogenous base. Pharmaceutically acceptable phospholipids can include without limitation phosphatidylcholines, phosphatidylserines and phosphatidylethanolamines. In one embodiment the composition comprises phosphatidylcholine, derived for example from natural lecithin. Any source of lecithin can be used, including animal sources such as egg yolk, but plant sources are generally preferred. Soy is a particularly rich source of lecithin that can provide phosphatidylcholine for use in the present invention.

Illustratively, a suitable amount of phospholipid is about 15% to about 75%, for example about 30% to about 60%, by weight of the carrier, although greater and lesser amounts can be useful in particular situations.

Ingredients useful as components of the solubilizing agent are not particularly limited and will depend to some extent on the particular drug and the desired concentration of drug and of phospholipid. In one embodiment, the solubilizing agent comprises one or more glycols and/or one or more glyceride materials.

Suitable glycols include propylene glycol and polyethylene glycols (PEGs) having molecular weight of about 200 to about 1,000 g/mol. e.g., PEG 400, which has an average molecular weight of about 400 g/mol. Such glycols can provide relatively high solubility of the drug; however in some cases the drug, particularly a drug having a tendency for hydrolytic, solvolytic or oxidative instability, can exhibit chemical degradation to some degree when in solution in a carrier comprising such glycols. This can be evident by color changes of the drug solution with time. The higher the glycol content of the carrier, the greater may be the tendency for degradation of a chemically unstable drug. In one embodiment, therefore, one or more glycols are present in a total glycol amount of at least about 1% but less than about 50%, for example less than about 30%, less than about 20%, less than about 15% or less than about 10% by weight of the carrier. In another embodiment, the carrier comprises substantially no glycol.

Suitable glyceride materials include, without limitation, medium to long chain mono-, di- and triglycerides. The term “medium chain” herein refers to hydrocarbyl chains individually having more than about 6 and less than about 12 carbon atoms, including for example C₈ to C₁₀ chains. Thus glyceride materials comprising caprylyl and capryl chains, e.g. caprylic/capric mono-, di- and triglycerides, are examples of “medium chain” glyceride materials herein. The term “long chain” herein refers to hydrocarbyl chains individually having at least about 12, for example about 12 to about 18, carbon atoms, including for example lauryl, myristyl, cetyl, stearyl, oleyl, linoleyl and linolenyl chains. Medium to long chain hydrocarbyl groups in the glyceride materials can be saturated, mono- or polyunsaturated.

In another embodiment the carrier comprises Gelucire® 44/14. Gelucire® 44/14 is a semisolid excipient consisting of 20% mono-, di-, and tri-glycerides and 72% mono- and di-fatty acid esters of PEG 1500 and 8% free PEG 1500. It acts as an emulsifier and solvent for many drugs and is used to enhance bioavailability by improving solubility.

In one embodiment the carrier comprises, as a major component of the solubilizing agent, a medium chain and/or a long chain triglyceride material. A suitable example of a medium chain triglyceride material is a caprylic/capric triglyceride product such as, for example, Captex 355 EP™ of Abitec Corp. and products substantially equivalent thereto. Suitable examples of long chain triglycerides include any pharmaceutically acceptable vegetable oil, for example canola, coconut, corn, flaxseed, safflower, soy and sunflower oils, and mixtures of such oils.

Where one or more glyceride materials are present as a major component of the solubilizing agent, a suitable total amount of glycerides is an amount effective to solubilize the phospholipid and, in combination with other components of the carrier, effective to maintain the drug in solution. For example, glyceride materials such as medium chain and/or long chain triglycerides can be present in a total glyceride amount of about 5% to about 70%, for example about 15% to about 60% or about 25% to about 50%, by weight of the carrier. Additional solubilizing agents that are other than glycols or glyceride materials can be included if desired. Some of these agents, for example vinylpyrrolidone dimer (1,3-bis-(pyrrolidon-1-yl)-butan, or VP dimer), is a new synthetic excipient that is often used as a solvent for poorly water soluble compounds. Other examples of such agents, for example N-substituted amide solvents such as dimethylformamide (DMF) and N,N-dimethylacetamide (DMA), can, in specific cases, assist in raising the limit of solubility of the drug in the carrier, thereby permitting increased drug loading. However, N-substituted amides including DMF and DMA can present regulatory and/or toxicological issues that restrict the amount of such solvents that can be used in a formulation. Furthermore, the carriers useful herein generally provide adequate solubility of small-molecule drugs of interest herein without such additional agents. Accordingly, in one embodiment a drug loading of at least about 67 mg/ml is achieved in a carrier comprising substantially no N-substituted amide solvent, for example less than about 2 mg/ml, or less than about 1 mg/ml, of such a solvent.

Even when a sufficient amount of a glycol or glyceride material is present to solubilize the phospholipid, the resulting carrier solution and/or the drug-carrier system may be rather viscous and difficult or inconvenient to handle. In such cases it may be found desirable to include in the carrier a viscosity reducing agent in an amount effective to provide acceptably low viscosity. An example of such an agent is ethanol, preferably introduced in a form that is substantially free of water, for example 99% ethanol or absolute ethanol. Excessively high concentrations of ethanol should, however, generally be avoided. This is particularly true where, for example, the drug-carrier system is to be administered in a gelatin capsule, because of the tendency of high ethanol concentrations to result in mechanical failure of the capsule. In general, suitable amounts of ethanol are 0% to about 25%, for example about 1% to about 20% or about 3% to about 15%, by weight of the carrier. Optionally, the carrier further comprises a pharmaceutically acceptable non-phospholipid surfactant. One of skill in the art will be able to select a suitable surfactant for use in a composition of the invention. Illustratively, a surfactant such as polysorbate 80 can be included in an amount of 0% to about 5%, for example 0% to about 2% or 0% to about 1%, by weight of the carrier. Also, a surfactant such as polysorbate 20 can be included in an amount of 0% to about 25%, for example 0% to about 10%, for example 0% to about 5%, or 0% to about 2%, by weight of the carrier.

Another example of a non-phospholipid surfactant comprised in the present invention is Vitamin E TPGS, d-α-tocopheryl polyethylene glycol 1000 succinate, which is a water-soluble derivative of natural-sourced Vitamin E. Structurally it comprises a dual nature of lipophilicity and hydrophilicity, similar to a surface active agent. Due to its solubilization capacity for lipophilic compounds and its surfactant-like property, it is recommended for use in dosage forms as an emulsifier, solubilizer and absorption enhancer.

Other ingredients can optionally be present in the carrier, selected for example from conventional formulation ingredients such as antioxidants, preservatives, colorants, flavorants and combinations thereof. As indicated above, the carrier can optionally comprise a solid or semi-solid substrate having the drug solution adsorbed therein or thereon. Examples of such substrates include particulate diluents such as lactose, starches, silicon dioxide, etc., and polymers such as polyacrylates, high molecular weight PEGs, or cellulose derivatives, e.g. hydroxypropylmethylcellulose (HPMC). Where a solid solution is desired, a high melting point ingredient such as a wax can be included. A solid drug-carrier system can optionally be encapsulated or, if desired, delivered in tablet form. The drug-carrier system can, in some embodiments, be adsorbed on, or impregnated into, a drug delivery device.

Conveniently, pre-blended products are available containing a suitable phospholipid + solubilizing agent combination for use in compositions of the present invention. It is emphasized that, while compositions comprising such products are embraced by the present invention, no limitation to such compositions is intended. Pre-blended phospholipid + solubilizing agent products can be advantageous in improving ease of preparation of the present compositions.

An illustrative example of a pre-blended phospholipid + solubilizing agent product is Phosal 50 PG™, available from American Lecithin Co. of Oxford, Conn., which comprises, by weight, not less than 50% phosphatidylcholine, not more than 6% lysophosphatidylcholine, about 35% propylene glycol, about 3% mono- and diglycerides from sunflower oil, about 2% soy fatty acids, about 2% ethanol, and about 0.2% ascorbyl palmitate.

Another illustrative example is Phosal 53 MCT™, also available from American Lecithin Co., which contains, by weight, not less than 53% phosphatidylcholine, not more than 6% lysophosphatidylcholine, about 29% medium chain triglycerides, 3-6% (typically about 5%) ethanol, about 3% mono- and diglycerides from sunflower oil, about 2% oleic acid, and about 0.2% ascorbyl palmitate.

Yet another illustrative example is Phosal 50 SA+™, also available from American Lecithin Co., which contains, by weight, not less than 50% phosphatidylcholine and not more than 6% lysophosphatidylcholine in a solubilizing system comprising safflower oil and other ingredients.

The phosphatidylcholine component of each of these pre-blended products is derived from soy lecithin. Substantially equivalent products may be obtainable from other suppliers.

A pre-blended product such as Phosal 50 PG™, Phosal 53 MCT™ or Phosal 50 SA+™ can, in some embodiments, constitute substantially the entire carrier system for a drug of low water solubility. In other embodiments, additional ingredients are present, for example ethanol (additional to any that may be present in the pre-blended product), non-phospholipid surfactant such as polysorbate 80, polyethylene glycol and/or other ingredients. Such additional ingredients, if present, are typically included in only minor amounts. Illustratively, Phosal 53 MCT™ or a pre-blended product substantially equivalent thereto can be included in the carrier in an amount of about 50% to 100%, for example about 80% to 100%, by weight of the carrier.

In embodiments of the invention as described above, the drug-carrier system is dispersible in an aqueous phase to form a non-gelling, substantially non-transparent liquid dispersion. This property can readily be tested by one of skill in the art, for example by adding 1 part of the drug-carrier system to about 20 parts of water with agitation at ambient temperature and assessing gelling behavior and transparency of the resulting dispersion. Compositions having ingredients in relative amounts as indicated herein will generally be found to pass such a test, i.e., to form a liquid dispersion that does not gel and is substantially non-transparent. The requirement herein for “non-gelling” behavior removes from the scope of the invention compositions containing, in addition to components specified herein, a gel-promoting agent in a gel-promoting effective amount. The requirement herein for a “substantially non-transparent” dispersion on mixing with an aqueous phase is believed to be satisfied by compositions as described above having any substantial amount of the phospholipid component, although for clarification it is emphasized that the compositions themselves, being substantially non-aqueous, are generally clear and transparent. In this regard, it is noted that phospholipids tend to form bi- and multilamellar aggregates when placed in an aqueous environment, such aggregates generally being large enough to scatter transmitted light and thereby provide a non-transparent, e.g. cloudy, dispersion. In the case of Phosal 53 MCT™, for example, dispersion in an aqueous environment typically forms not only multilamellar aggregates but also a coarse oil-in-water emulsion. Presence of multilamellar aggregates can often be confirmed by microscopic examination in presence of polarized light, such aggregates tending to exhibit birefringence, for example generating a characteristic “Maltese cross” pattern.

Without being bound by theory, it is believed that behavior of the drug-carrier system of a composition of the invention upon mixing with an aqueous phase is indicative of how the composition interacts with gastrointestinal fluid following oral administration to a subject. Although formation of a gel can be useful for controlled-release topical delivery of a drug, for example to the periodontal region of the mouth as mentioned in above-cited U.S. Pat. No. 6,464,987, it is believed that gelling would be detrimental to efficient gastrointestinal absorption. For this reason, embodiments of the invention described above specify a composition comprising a drug-carrier system that does not gel when mixed with an aqueous phase. It is further believed, again without being bound by theory, that formation of bi- and multilamellar aggregates in the gastrointestinal fluid, as evidenced by non-transparency of the dispersion formed upon mixing the drug-carrier system with an aqueous phase, can be an important factor in providing the relatively high bioavailability of certain compositions of the invention when administered orally.

Illustratively where the drug is ABT-869, the carrier ingredients and amounts thereof are selected to provide solubility of the drug in the carrier of at least about 10 mg/ml, for example at least about 50 mg/ml, at least about 67 mg/ml or at least about 100 mg/ml, at about 25° C. As another example, where the drug is ABT-102, the carrier ingredients and amounts thereof are selected to provide solubility of the drug in the carrier of at least about 10 mg/ml, for example at least about 50 mg/ml, at least about 100 mg/ml, at least about 150 mg/ml, or at least about 200 mg/ml at about 25° C.

In certain embodiments, the carrier ingredients and amounts thereof are selected to provide enhanced bioabsorption by comparison with a standard solution of the drug, e.g., a solution in PEG 400, when administered orally. Such enhanced bioabsorption can be evidenced by a pharmacokinetic profile having one or more of a higher Cₘₐₓ, a shorter Tₘₐₓ, or an increased bioavailability as measured by AUC, for example AUC₀₋₂₄ or AUC_{0-∞}. Illustratively, bioavailability can be expressed as a percentage, for example using the parameter F, which computes AUC for oral delivery of a test composition as a percentage of AUC for intravenous (IV) delivery of the drug in a suitable solvent, taking into account any difference between oral and IV doses.

Bioavailability can be determined by pharmacokinetic studies in humans or in any suitable model species. For present purposes, a dog model, as illustratively described in Example 5 below, is generally suitable. In various illustrative embodiments, where the drug is ABT-869, compositions of the invention exhibit oral bioavailability of at least about 20%, for example at least about 25%, at least about 30%, at least about 35%, at least about 40%, at least about 45% or at least about 50%, in a dog model.

In one example, the composition comprises ABT-869 and a carrier comprising ingredients and amounts thereof selected to provide (a) solubility of ABT-869 of at least about 50 mg/ml at about 25° C.; and (b) a pharmacokinetic profile upon oral administration of the composition in a dog model exhibiting a bioavailability of at least about 25%. In another example, the composition comprises ABT-869 and a carrier comprising ingredients and amounts thereof selected to provide (a) solubility of ABT-869 of at least about 67 mg/ml at about 25° C.; and (b) a pharmacokinetic profile upon oral administration of the composition in a dog model exhibiting a bioavailability of at least about 30%.

In yet another example, the composition comprises ABT-869 and a carrier comprising ingredients and amounts thereof selected to provide (a) solubility of ABT-869 of at least about 100 mg/ml at about 25° C.; and (b) a pharmacokinetic profile upon oral administration of the composition in a dog model exhibiting a bioavailability of at least about 50%.

In another example, the composition comprises ABT-102 and a carrier comprising ingredients and amounts thereof selected to provide a pharmacokinetic profile exhibiting a bioavailability of at least 30% upon oral administration of the composition in a dog model.

The present invention is not limited by the process used to prepare a composition as embraced or described herein. Any suitable process of pharmacy can be used. Illustratively, compositions of the invention can be prepared by a process comprising simple mixing of the recited ingredients, wherein order of addition is not critical, to form a drug-carrier system. It is noted, however, that if the phospholipid component is used in its solid state, for example in the form of soy lecithin, it will generally be desirable to first solubilize the phospholipid with the solubilizing agent component or part thereof. Thereafter other ingredients of the carrier, if any, and the drug can be added by simple mixing, with agitation as appropriate. As mentioned above, use of a pre-blended product comprising phospholipid and solubilizing agent can simplify preparation of the composition. An illustrative process employing such a product, in this case Phosal 53 MCT™, is presented in Example 3 below. Optionally, the drug-carrier system can be used as a premix for capsule filling, as illustrated in Example 4 below. The term “filling” used in relation to a capsule herein means placement of a desired amount of a composition in a capsule shell, and should not be taken to mean that all space in the capsule is necessarily occupied by the composition.

Compositions embraced herein, including compositions described generally or with specificity herein, are useful for orally delivering a drug of low water solubility to a subject. Accordingly, a method of the invention for delivering a drug of low water solubility to a subject comprises orally administering a composition as described herein.

The subject can be human or non-human (e.g., a farm, zoo, work or companion animal) but is typically a human patient in need of the drug to prevent or treat a disease, disorder or condition for which the drug is indicated.

The composition can be administered in an amount providing a therapeutically effective dose of the drug. What constitutes a therapeutically effective dose depends on the particular drug, the subject (including species and body weight of the subject), the disease, disorder or condition to be prevented or treated, and other factors, and can accordingly vary within wide margins, for example from about 0.01 to about 1,000 mg. It will be understood that recitation herein of a “therapeutically effective” dose herein does not necessarily require that the drug be therapeutically effective if only a single such dose is administered; typically therapeutic efficacy depends on the composition being administered repeatedly according to a regimen involving adequate frequency and duration of administration.

Where the composition is the “semi-solid capsule”, it means that the drug carrier system is semisolid and is filled into capsules. These semisolid filled capsules can be swallowed whole, typically with the aid of water or other imbibable liquid. It is understood that “imbibable” means consumable.

Where the composition is the “semi-solid formulation”, it means that the drug carrier system is semisolid and requires to be either filled into a capsule prior to administration or melted and administered by gavage at a temperature of about 37° C. It is understood that “gavage” means introduced in the stomach by means of a tube.

Where the composition is in the form of an unencapsulated liquid, the composition can be swallowed neat, but administration is generally more convenient and pleasant if the composition is first diluted in a suitable imbibable liquid. Suitable liquid diluents include without limitation any aqueous beverage such as water, milk, fruit juice (e.g., apple juice, grape juice, orange juice, etc.), carbonated drink, enteral nutrition formula, energy drink, tea or coffee. Where a liquid diluent is to be used, the composition should be mixed with the diluent using sufficient agitation (e.g., by shaking and/or stirring) to thoroughly disperse the composition in the diluent, and administered immediately thereafter, so that the composition does not separate from the diluent before swallowing. Any convenient rate of dilution can be employed, for example about 1 to about 100, or about 5 to about 50, parts by volume of the composition per part by volume of the diluent.

Where the composition is in the form of a capsule, one to a small plurality of capsules can be swallowed whole, typically with the aid of water or other imbibable liquid to help the swallowing process. Suitable capsule shell materials include, without limitation, gelatin (in the form of hard gelatin capsules or soft elastic gelatin capsules), starch, carrageenan and HPMC. Where the drug-carrier system is liquid, soft elastic gelatin capsules are generally preferred.

Where the small-molecule drug of low water solubility is a compound of formula (I) or formula (II) above, illustratively ABT-869, it is preferred but not essential that the drug-carrier system have the properties of being substantially non-gelling and substantially non-transparent upon dispersion in an aqueous phase, as defined above.

In various embodiments of the invention, a method is provided for treating a condition in a subject for which a PTK inhibitor is indicated. Such a method comprises administering to the subject, by a suitable route of administration, a composition as described generally or with specificity herein having as the drug of low water solubility a compound of formula (I) above. The drug can be, for example, a compound of formula (II) above, including one wherein X is NH; R¹, R², R³ and R⁴ are each hydrogen; L is NHC(O)NH; and R¹⁰ and R¹¹ are independently selected from the group consisting of hydrogen, alkyl and halo. In one embodiment, the drug is ABT-869.

A preferred route of administration is oral. Oral administration can be of a neat or diluted drug-carrier system, particularly where the drug-carrier system is liquid, or a capsule, for example a liquid-filled capsule, as described above.

The condition to be treated by the present method can include any disease or disorder for which a PTK inhibitor is indicated, for example macular degeneration or any condition that involves neoplasia. Such conditions illustratively include acute myelogenous leukemia, colorectal cancer, non-small cell lung cancer, hepatocellular carcinoma, non-Hodgkin's lymphoma, ovarian cancer, breast cancer, prostate cancer and kidney cancer.

Suitable doses of ABT-869 are generally about 1 to about 500 mg, more typically about 10 to about 300 mg or about 20 to about 200 mg, for example about 50 to about 100 mg, administered at a frequency of about once a week to about four times a day. In most cases a frequency of administration of about once to about twice a day is suitable.

Where the small-molecule drug of low water solubility is a compound of formula (III) or formula (IV) above, illustratively ABT-102, it is preferred but not essential that the drug-carrier system have the properties of being substantially non-gelling and substantially non-transparent upon dispersion in an aqueous phase, as defined above.

In various embodiments of the invention, a method is provided for treating a condition in a subject for which a TRPV1 antagonist is indicated. Such a method comprises administering to the subject, by a suitable route of administration, a composition as described generally or with specificity herein having as the drug of low water solubility a compound of formula (III) above. The drug can be, for example, a compound of formula (IV) above, such as ABT-102.

A preferred route of administration is oral. Oral administration can be of a neat or diluted drug-carrier system, particularly where the drug-carrier system is liquid, or a capsule, for example a liquid-filled capsule, as described above.

The condition to be treated by the present method can include any disease or disorder for which a TRPV1 antagonist is indicated, for example urinary disorders or any condition that involves pain. Such conditions illustratively include urinary dysfunction, bladder overeactivity, urinary incontinence, neuropathic pain, pain associated with inflammatory states, and migraine.

### EXAMPLES

The following examples are merely illustrative, and do not limit this disclosure in any way. Trademarked ingredients used in the examples can be substituted with comparable ingredients from other suppliers. Where a pre-blended product such as Phosal 50 PG™, Phosal 53 MCT™ or Phosal 50 SA+™ is indicated below, its components can, if desired, be added individually rather than in the form of the pre-blended product. Composition of each of Phosal 50 PG™, Phosal 53 MCT™ and Phosal 50 SA+™ is given above. Other trademarked ingredients used in the examples include:
Captex 355 EP™ of Abitec Corp.: caprylic/capric triglycerides
Tween 80™ of Uniqema: polysorbate 80 surfactant.
Gelucire™ 44/14 of Gattefossé: lauroyl macrogol glycerides.
Labrasol™ of Gattefossé: Caprylocapryl Polyoxyglycerides
Cremophor EL™ of BASF: polyoxyl 35 castor oil
Tween 20™ of Uniquema: polysorbate 20 surfactant.

The examples below illustrate aspects of the invention and demonstrate, inter alia, that a liquid carrier comprising a phospholipid and a pharmaceutically acceptable solubilizing agent can provide acceptable solubility and/or bioavailability of a drug of low water solubility such as ABT-869, isotretinoin or paricalcitol formulated in solution in such a carrier. All references cited above are incorporated herein by reference in their entirety. Percentage amounts herein are by weight unless otherwise specified. The words “comprise”, “comprises”, and “comprising” are to be interpreted inclusively rather than exclusively.

### Example 1

### Screening of Carriers for Solubility of ABT-869

Approximately 20 mg of ABT-869 was weighed and added to a 0.3 ml vial. A test carrier (100 μl) was then added by pipette to the vial. The vial was three times alternately vortexed for about 30 seconds and sonicated for about 1 minute to ensure adequate wetting and dispersion of the ABT-869. The vial was wrapped in aluminum foil, placed in a Labquake™ rotator and rotated for a minimum of 24 hours. After 24 hours, contents of the vial were observed for the presence of solid ABT-869. If solid was still present, carrier was added until all solid had dissolved and the resulting solution was clear. Approximate solubility is reported in Table 1 below as a range based on volume of carrier providing a clear solution and volume of carrier where solid was present. All solubility values were determined at room temperature.

TABLE 1
Solubility of ABT-869 in different carriers

| Carrier | | Solubility (S) |
|---|---|---|
| (% by weight) | | (mg/ml) |
| 100% | PEG 400 | S > 200 |
| 10% | ethanol USP, absolute | S > 200 |
| 90% | PEG 400 | |
| 10% | ethanol USP, absolute | S > 200 |
| 20% | polysorbate 80 | |
| 70% | PEG 400 | |
| 10% | ethanol USP, absolute | S > 200 |
| 30% | Phosal 50 PG ™ | |
| 60% | PEG 400 | |
| 10% | ethanol USP, absolute | 50 < S < 67 |
| 90% | Phosal 50 PG ™ | |
| 10% | ethanol USP, absolute | 67 < S < 100 |
| 90% | Phosal 53 MCT ™ | |
| 100% | Captex 355 EP ™ | S < 50 |

The results of this screening study gave preliminary indication that carriers comprising Phosal 50 PG™ or Phosal 53 MCT™ could be useful for preparing formulations of ABT-869 at a drug concentration of at least about 50 mg/ml.

### Example 2

### Solubility of ABT-869 in Carriers Comprising Phosal 53 MCT™

Solubility of ABT-869 was measured in various carriers comprising Phosal 53 MCT™. Approximately 100-400 mg of ABT-869 was weighed and added to a 4 ml glass vial, to which 2 ml of a test carrier was added. The vial was then vortexed and sonicated for 10 minutes. The vials were wrapped with aluminum foil, placed in a water bath at 25° C. and agitated for 2 days. The contents of the vials were then filtered and the filtrate diluted 25× with mobile phase for HPLC analysis. Results are presented in Table 2.

TABLE 2
Solubility of ABT-869 in various carriers

| Carrier (% by weight) | | Solubility (mg/g) |
|---|---|---|
| 100% | Phosal 53 MCT ™ | 95 |
| 5% | ethanol USP, absolute | 115 |
| 95% | Phosal 53 MCT ™ | |
| 10% | ethanol USP, absolute | 97 |
| 90% | Phosal 53 MCT ™ | |
| 10% | PEG 400 | 139 |
| 90% | Phosal 53 MCT ™ | |
| 20% | PEG 400 | 166 |
| 80% | Phosal 53 MCT ™ | |
| 30% | PEG 400 | 185 |
| 70% | Phosal 53 MCT ™ | |
| 40% | PEG 400 | 199 |
| 60% | Phosal 53 MCT ™ | |
| 50% | PEG 400 | 221 |
| 50% | Phosal 53 MCT ™ | |
| 10% | PEG 400 | >123 |
| 5% | ethanol USP, absolute | |
| 85% | Phosal 53 MCT ™ | |
| 10% | PEG 400 | >122 |
| 0.5% | Tween 80 ™ | |
| 4.5% | ethanol USP, absolute | |
| 85% | Phosal 53 MCT ™ | |

Results showed that addition of 5% ethanol to Phosal 53 MCT™ (which already contains about 5% ethanol) enhanced ABT-869 solubility over Phosal 53 MCT™ alone. Substitution of PEG 400 for ethanol gave further improvement in solubility, which increased with increasing PEG 400 concentration in the carrier.

### Example 3

### Preparation of an Illustrative Liquid Pharmaceutical Composition

Preparation of carrier. Phosal 53 MCT™ (18.02 g) and ethanol USP, absolute (2.01 g) were weighed and added to a 30 ml amber bottle. The bottle was agitated by hand until a uniform carrier mixture consisting of 10 parts ethanol and 90 parts Phosal 53 MCT™ was obtained.

Preparation of pharmaceutical composition. A 9.36 g aliquot of the carrier mixture prepared as above was weighed and added to a 20 ml amber vial along with a stir bar. ABT-869 (0.64 g) was added to the vial with stirring until the ABT-869 was completely dissolved. The resulting solution, containing 6.4% by weight ABT-869, was clear and yellow.

If desired, the pharmaceutical composition can be prepared under a nitrogen blanket to minimize any risk of loss of potency through instability of the drug during formulation.

### Example 4

### Preparation of an Illustrative Encapsulated Pharmaceutical Composition

The solution prepared in Example 3 was used as a premix for preparing an encapsulated pharmaceutical composition. Soft elastic gelatin capsules were individually filled with 781 mg (target fill weight) of the premix, providing a 50 mg ABT-869 dose per capsule. The capsules were filled using a syringe/needle combination and subsequently heat-sealed.

### Example 5

### Pharmacokinetic Study

An ABT-869 composition of the invention (Formulation #2) and a comparative composition (Formulation #1) were evaluated in a pharmacokinetic study in fasted dogs. Formulation #1 was a liquid composition comprising PEG 400 having ABT-869 in solution therein at a concentration of 20 mg/ml. Formulation #2 was in the form of soft elastic gelatin capsules each containing 50 mg ABT-869 (6.4% by weight ABT-869 in a carrier solution), prepared as described in Examples 3 and 4 above. The carriers were as follows:

Formulation #1:
100% PEG 400

Formulation #2:
10% ethanol USP, absolute
90% Phosal 53 MCT™

Formulation #1 was administered by oral gavage to 3 dogs in an amount of 0.5 ml/kg BW (body weight), calculated to provide an ABT-869 dose of 10 mg/kg BW. Formulation #2 was administered orally at a dose of 100 mg (two 50 mg capsules) per dog to 6 dogs, a dose equivalent on average to 10.8 mg/kg BW. Both formulations were administered under fasting conditions. Blood plasma samples were taken before dosing (time 0) and at 0.25, 0.5, 1, 1.5, 2, 3, 4, 6, 9, 12, 15 and 24 hours after dosing. ABT-869 concentrations in each plasma sample were determined by HPLC-MS. Pharmacokinetic (PK) parameters calculated from the data are presented in Table 3. Bioavailability was determined as the parameter F, by comparison with intravenous administration of ABT-869 in a PEG 400 solution in a separate group of dogs.

TABLE 3
PK parameters for Formulations #1 and #2

| | ABT-869 | | | | | | |
|---|---|---|---|---|---|---|---|
| | dose | Cₘₐₓ | Tₘₐₓ | T_{1/2} | AUC_{0-∞} | | |
| Form. | (mg/kg BW) | (μg/ml) | (hr) | (hr) | (μg · hr/ml) | F (%) | n |
| #1 | 10 | 0.78 | 2.7 | 1.5 | 4.40 | 18.9 | 3 |
| #2 | 10.8 | 1.69 | 1.4 | 1.5 | 8.13 | 37.7 | 6 |

As shown in Table 3, Formulation #2 of the invention provided substantially higher bioavailability of ABT-869 than the simple PEG 400 solution (Formulation #1).

### Example 6

### Pharmacokinetic Study

Three ABT-869 compositions of the invention (Formulations #3, #4 and #5) were evaluated in a pharmacokinetic study in fasted dogs. All were in the form of soft gelatin capsules each containing 75 mg ABT-869 (7.5% by weight ABT-869 in a carrier solution), prepared substantially as described in Examples 3 and 4 above. The carriers were as follows:

Formulation #3:
10% PEG 400
90% Phosal 53 MCT™

Formulation #4:
10% PEG 400
0.5% Tween 80™
89.5% Phosal 53 MCT™

Formulation #5:
5% ethanol USP, absolute
95% Phosal 53 MCT™

Each composition was administered orally to 3 dogs at an ABT-869 dose of 75 mg per dog. Blood plasma samples were taken before dosing (time 0) and at 0.25, 0.5, 1, 1.5, 2, 3, 4, 6, 9 and 12 hours after dosing. ABT-869 concentrations in each plasma sample were determined and PK parameters calculated from the data as in Example 5. PK parameters are presented in Table 4.

TABLE 4
PK parameters for Formulations #3, #4 and #5

| | ABT-869 | | | | | | |
|---|---|---|---|---|---|---|---|
| | dose | Cₘₐₓ | Tₘₐₓ | T_{1/2} | AUC_{0-∞} | | |
| Form. | (mg/dog) | (μg/ml) | (hr) | (hr) | (μg · hr/ml) | F (%) | n |
| #3 | 75 | 1.94 | 1.7 | 1.5 | 10.42 | 59.5 | 3 |
| #4 | 75 | 2.08 | 2.3 | 1.5 | 9.43 | 53.7 | 3 |
| #5 | 75 | 1.52 | 2.5 | 1.6 | 6.37 | 38.5 | 3 |

Compositions having 10% PEG 400 together with Phosal 53 MCT™ in the carrier (Formulations #3 and #4) exhibited higher bioavailability of ABT-869 than the composition having 10% ethanol (Formulation #2) in the study of Example 5 above. Reducing ethanol to 5% (Formulation #5) did not substantially affect bioavailability when compared with Formulation #2 in Example 5. Reduction of ethanol in a soft gelatin capsule composition could be advantageous in minimizing risk of capsule failure.

### Example 7

### Pharmacokinetic Study

Two ABT-869 compositions of the invention (Formulations #6 and #7) were evaluated in a pharmacokinetic study in fasted dogs. Both were in the form of soft gelatin capsules each containing 100 mg ABT-869 (10% by weight ABT-869 in a carrier solution), prepared substantially as described in Examples 3 and 4 above. The carriers were as follows:

Formulation #6:
20% PEG 400
80% Phosal 50 PG™

Formulation #7:
10% PEG 400
90% Phosal 53 MCT™

Each composition was administered orally to 3 dogs at an ABT-869 dose of 100 mg per dog. Blood plasma samples were taken before dosing (time 0) and at 0.25, 0.5, 1, 1.5, 2, 3, 4, 6, 9, 12, 15 and 24 hours after dosing. ABT-869 concentrations in each plasma sample were determined and PK parameters calculated from the data as in Example 5. PK parameters are presented in Table 5.

TABLE 5
PK parameters for Formulations #6 and #7

| | ABT-869 | | | | | | |
|---|---|---|---|---|---|---|---|
| | dose | Cₘₐₓ | Tₘₐₓ | T_{1/2} | AUC_{0-∞} | | |
| Form. | (mg/dog) | (μg/ml) | (hr) | (hr) | (μg · hr/ml) | F (%) | n |
| #6 | 100 | 0.89 | 1.7 | 1.5 | 3.32 | 15.5 | 3 |
| #7 | 100 | 1.57 | 1.5 | 1.6 | 6.43 | 27.4 | 3 |

In this study, Formulation #6, comprising Phosal 50 PG™ (having propylene glycol as the primary solubilizing agent within the pre-blended product) exhibited lower bioavailability than Formulation #7, comprising Phosal 53 MCT™ (having medium chain triglycerides as the primary solubilizing agent within the pre-blended product).

### Example 8

### Pharmacokinetic Study

Three ABT-869 compositions of the invention (Formulations #8, #9 and #10) were evaluated in a pharmacokinetic study in fasted dogs. All were in the form of soft gelatin capsules each containing 100 mg ABT-869 (7.5% by weight ABT-869 in a carrier solution), prepared substantially as described in Examples 3 and 4 above. The carriers were as follows:

Formulation #8:
10% PEG 400
90% Phosal 53 MCT™

Formulation #9:
10% PEG 400
5% ethanol USP, absolute
85% Phosal 53 MCT™

Formulation #10:
10% PEG 400
0.5% Tween 80™
4.5% ethanol USP, absolute
85% Phosal 53 MCT™

Each composition was administered orally to 3 dogs at an ABT-869 dose of 100 mg per dog. Blood plasma samples were taken before dosing (time 0) and at 0.25, 0.5, 1, 1.5, 2, 3, 4, 6, 9 and 12 hours after dosing. ABT-869 concentrations in each plasma sample were determined and PK parameters calculated from the data as in Example 5. PK parameters are presented in Table 6.

TABLE 6
PK parameters for Formulations #8, #9 and #10

| | ABT-869 | | | | | | |
|---|---|---|---|---|---|---|---|
| | dose | Cₘₐₓ | Tₘₐₓ | T_{1/2} | AUC_{0-∞} | | |
| Form. | (mg/dog) | (μg/ml) | (hr) | (hr) | (μg · hr/ml) | F (%) | n |
| #8 | 100 | 2.31 | 1.3 | 1.7 | 10.93 | 46.2 | 3 |
| #9 | 100 | 1.67 | 1.5 | 1.6 | 7.28 | 30.9 | 3 |
| #10 | 100 | 2.90 | 1.7 | 1.8 | 15.62 | 67.7 | 3 |

Addition of Tween 80™ to the carrier (Formulation #10) appeared to improve bioavailability in this study by comparison with Formulation #9.

### Example 9

### Pharmacokinetic Study in Fasted and Non-Fasted Dogs and Comparison of Administration in Encapsulated and Diluted Liquid Dosage Form

An ABT-869 composition of the invention (Formulation #11) was evaluated in a pharmacokinetic study in fasted and non-fasted dogs to evaluate effect of food. The composition, having a 50 mg/ml ABT-869 loading, was administered as gelatin capsules providing a dosage volume of 2 ml/dog, for an ABT-869 dosage of 100 mg/dog (equivalent on average to 9.8 mg/kg BW). The formulation was prepared substantially as described in Examples 3 and 4 above.

In another study, Formulation #11 was tested in liquid form, diluted in either apple juice or an enteral nutrition formula (Ensure Plus™ of Abbott Laboratories), at the same dosage. The liquid composition was administered by oral gavage at a 1:20 dilution in the apple juice or nutrition formula.

The carrier was as follows:

Formulation #11:
10% ethanol USP, absolute
0.5% Tween 80™
89.5% Phosal 53 MCT™

For both studies, the compositions were administered using a two-period crossover design in a group of 6 dogs. Blood plasma samples were taken before dosing (time 0) and at 0.25, 0.5, 1, 1.5, 2, 3, 4, 6, 9, 12, 15 and 24 hours after dosing. ABT-869 concentrations in each blood sample were determined and PK parameters calculated from the data as in Example 5. PK parameters are presented in Table 7.

TABLE 7
Effect of food and dosage form on PK parameters for Formulation #11

| | ABT-869 | | | | | | |
|---|---|---|---|---|---|---|---|
| | dose | Cₘₐₓ | Tₘₐₓ | T_{1/2} | AUC_{0-∞} | | |
| Form. | (mg/dog) | (μg/ml) | (hr) | (hr) | (μg · hr/ml) | F (%) | n |
| #11 (capsule, fasted) | 100 | 3.28 | 2.1 | 1.5 | 14.60 | 61.5 | 6 |
| #11 (capsule, with food) | 100 | 1.68 | 2.3 | 1.4 | 7.29 | 30.6 | 6 |
| #11 (liquid, in apple juice) | 100 | 2.20 | 1.8 | 1.3 | 9.82 | 41.2 | 6 |
| #11 (liquid, in Ensure Plus ™) | 100 | 2.14 | 1.9 | 1.4 | 9.98 | 41.9 | 6 |

Bioavailability of Formulation #11 administered in capsule form to non-fasted dogs was lower than when administered to fasted dogs.

Bioavailability of Formulation #11, when administered prediluted in either apple juice or nutrition formula, was intermediate between that of the same formulation administered in capsule form to fasted and non-fasted dogs.

### Example 10

### ABT-869 Formulation #12

A liquid ABT-869 composition of the invention (Formulation #12) was prepared substantially as described in Example 3 above. The composition consisted of the following ingredients:

| | |
|---|---|
| ABT-869 | 5.18% |
| Phosal 53 MCT ™ | 89.60% |
| ethanol USP, absolute | 4.74% |
| polysorbate 80 | 0.47% |

Formulation #12 was estimated to have at least a 6 months expiration date when stored at 5° C. protected from light.

### Example 11

### Isotretinoin Composition

Isotretinoin (a compound having a molecular weight of 300.43 g/mol and aqueous solubility of about 5 μg/ml) was tested for solubility in Phosal 53 MCT™ and found to have a solubility limit of 72-78 mg/g at 25° C. This is much greater than the solubility of isotretinoin found in typical solvent systems including ethanol (16.7 mg/g), caprylic/capric triglycerides (5.1 mg/g), oleic acid (19.1 mg/g) and soybean oil (2.4 mg/g).

An isotretinoin composition of the invention was prepared by adding to a 12 ml sample vial 6.58 g Phosal 53 MCT™ and 0.42 g isotretinoin. Six 4 mm glass beads were added and the vial was capped, wrapped with parafilm and aluminum foil, and placed on a Labquake™ rotator (8 rpm) at ambient temperature. When the drug was completely dissolved, the resulting drug-carrier system formed a clear, yellow, viscous liquid.

Hard gelatin capsules were prepared by filling the bottom half of each capsule with 666 mg (equivalent to 40 mg isotretinoin) of the drug-carrier system. Both halves of the capsule shell were assembled and sealed with a 20% by volume ethanol solution.

### Example 12

### Pharmacokinetic Study

In a pharmacokinetic study in fasted dogs, 6 dogs received oral administration of 40 mg isotretinoin as the formulation of Example 11 above (one capsule), by comparison with 30% wax formulations having drug particle sizes of 300, 180 or 75 μm, and also by comparison with two lots of Accutane™ soft gelatin capsules of Roche. Wax formulations can be prepared substantially as described in International Patent Publication No. WO 00/25772 of Hoffmann-La Roche AG, incorporated herein by reference in its entirety.

Blood plasma samples were taken before dosing (time 0) and at 0.25, 0.5, 1, 1.5, 2, 4, 6, 9, 12, 15 and 24 hours after dosing. Plasma concentrations of isotretinoin and its metabolite 4-oxoisotretinoin were determined by HPLC-MS, and then normalized for formulation potency. PK parameters were calculated and are presented in Tables 8 and 9 (ND=not determined).

TABLE 8
PK parameters for isotretinoin following oral administration of 40 mg
isotretinoin in dogs

| | | Isotretinoin | | | | |
|---|---|---|---|---|---|---|
| | Dose | Cₘₐₓ | AUC_{0-∞} | T_{1/2} | Tₘₐₓ | |
| Formulation | (mg/dog) | (ng/ml) | (ng · hr/ml) | (hr) | (hr) | n |
| Example 11 | 40 | 2,576 | 13,743 | 4.4 | 1.3 | 6 |
| wax, 300 μm | 40 | 644 | 3,969 | 6.7 | 1.8 | 6 |
| wax, 180 μm | 40 | 960 | 6,484 | 5.0 | 2.5 | 6 |
| wax, 75 μm | 40 | 1,284 | 8,460 | 5.0 | 2.2 | 6 |
| Accutane ™, | 40 | 1,364 | 8,102 | ND | 1.4 | 6 |
| lot 1 | | | | | | |
| Accutane ™, | 40 | 1,351 | 8,203 | ND | 1.9 | 6 |
| lot 2 | | | | | | |

TABLE 9
PK parameters for 4-oxoisotretinoin following oral administration of
40 mg sotretinoin in dogs

| | | 4-Oxoisotretinoin | | | | |
|---|---|---|---|---|---|---|
| | Dose | Cₘₐₓ | AUC_{0-∞} | T_{1/2} | Tₘₐₓ | |
| Formulation | (mg/dog) | (ng/ml) | (ng · hr/ml) | (hr) | (hr) | n |
| Example 11 | 40 | 33.6 | 317.7 | 5.2 | 6.2 | 6 |
| wax, 300 μm | 40 | 7.1 | 80.0 | 5.8 | 4.7 | 6 |
| wax, 180 μm | 40 | 13.0 | 128.7 | 6.0 | 5.7 | 6 |
| wax, 75 μm | 40 | 17.5 | 204.8 | 5.0 | 6.2 | 6 |
| Accutane ™, | 40 | 16.4 | 185.4 | ND | 5.1 | 6 |
| lot 1 | | | | | | |
| Accutane ™, | 40 | 14.3 | 164.5 | ND | 4.9 | 6 |
| lot 2 | | | | | | |

The composition of Example 11 of the present invention exhibited a higher Cₘₐₓ and a higher AUC_{0-∞}, for both isotretinoin and 4-oxoisotretinoin, than any of the comparative formulations tested.

### Example 13

### Solubility of Paricalcitol in Various Carriers

The vitamin D analog drug paricalcitol (a compound having a molecular weight of 416.63 g/mol and solubility in pH 7.4 buffer of 11.5 ng/ml) was the subject of a study comparing solubility in a variety of carriers. Equilibrium solubility was determined in duplicate after rotational agitation for 42 hours with excess drug. Mean solubility data are given in Table 10.

TABLE 10
Solubility of paricalcitol in various carriers (mean of 2 tests)

| Carrier | Solubility (μg/g) |
|---|---|
| oleic acid | 819 |
| medium chain monoglycerides (Capmul MCM ™) | 5,057 |
| glyceryl monooleate | 1,067 |
| medium chain triglycerides (Neobee M5 ™) | 165 |
| Neobee M5 ™ + 0.5% ethanol | 194 |
| castor oil | 344 |
| propylene glycol | 5,791 |
| PEG 400 | 1,085 |
| 10% hydroxypropyl-β-cyclodextrin in PEG | 734 |
| polysorbate 80 (Tween 80 ™) | 1,353 |
| triethyl citrate | 453 |
| Phosal 53 MCT ™ | 1,459 |
| Phosal 50 SA ™ | 752 |

Illustratively, solubility of paricalcitol in Phosal 53 MCT™ was relatively high by comparison with most carriers tested.

### Example 14

### Solubility of ABT-102 in Various Carriers

An accurately weighed quantity of about 1 g of each excipient was weighed into three glass vials. Semisolid excipients were warmed in a water bath at around 50-60° C. until completely melted before weighing. An accurately weighed quantity of ABT-102 of about 25 mg, 50 mg and 100 mg was weighed into each of the three vials containing the same excipients. The vials were closed tightly and mixed for around 30 seconds by vortexing and then sonicating in a warm water bath. The vials were visually observed for dissolution after 5-6 hours. Solubility is reported in Table 11 below based on volume of carrier providing a clear solution and volume of carrier where solid was present. All solubility values were determined at room temperature.

TABLE 11
Solubility of ABT-102 in different carriers

| | Solubility (S) |
|---|---|
| Carrier | (mg/ml) |
| PEG 400 | 71 |
| VP Dimer (VPD) | 160 |
| Vit.E TPGS | <200 |
| Phosal 50 PG ™ | 50 < S < 100 |
| Gelucire ® 44/14 | 25 < S < 50 |
| Phosal 53 MCT ™ | 50 < S < 100 |
| Polysorbate 20 | Not determined |
| Polysorbate 80 | Not determined |

### Example 15

### Pharmacokinetic Study. Formulations of ABT-102, 480 Mg Oral Dose in Dogs

| | |
|---|---|
| Formulation #13 | |
| Semi-solid formulation | 8% ABT-102; 25% TPGS; 32% Gelucire 44/14; |
| | 16% Phosal 50 PG; 19% VPD |
| Formulation #14 | |
| Semi-solid formulation | 6% ABT-102; 32% TPGS; 29% Gelucire 44/14; |
| | 15% Phosal 50 PG; 18% VPD |
| Formulation #15 | |
| Semi-solid formulation | 4% ABT-102; 52.8% TPGS; 28.8% |
| | Gelucire 44/14; 14.4% VPD |

TABLE 12
PK parameters for Formulations #13, #14, and #15

| | ABT-102 | | | | | | |
|---|---|---|---|---|---|---|---|
| | dose | Cₘₐₓ | Tₘₐₓ | T_{1/2} | AUC_{0-∞} | | |
| Form. | (mg/dog) | (μg/ml) | (hr) | (hr) | (μg · hr/ml) | F (%) | n |
| #13 | 480 | 4.09 | 4.7 | 3.0 | 39.47 | 42.3 | 3 |
| #14 | 480 | 4.40 | 5.3 | 2.2 | 35.61 | 38.7 | 3 |
| #15 | 480 | 4.21 | 5.3 | 2.2 | 38.60 | 41.2 | 3 |

### Example 16

### Pharmacokinetic study. Formulations of ABT-102, 640, 800 or 900 mg Oral Dose in Dogs

| | |
|---|---|
| Formulation #16 | |
| Semi-solid formulation | 4% ABT-102; 52.8% TPGS; 28.8% |
| | Gelucire 44/14; 14.4% VPD |
| Formulation #17 | |
| Semi-solid formulation | 5% ABT-102; 44% TPGS; 36% Gelucire 44/14; |
| | 15% VPD |
| Formulation #18 | |
| Semi-solid formulation | 8% ABT-102; 25% TPGS; 32% Gelucire 44/14; |
| | 16% Phosal 50PG; 19% VPD |

TABLE 13
PK parameters for Formulations #16, #17, and #18

| | ABT-102 | | | | | | |
|---|---|---|---|---|---|---|---|
| | dose | Cₘₐₓ | Tₘₐₓ | T_{1/2} | AUC_{0-∞} | | |
| Form. | (mg/dog) | (μg/ml) | (hr) | (hr) | (μg · hr/ml) | F (%) | n |
| #16 | 640 | 4.55 | 3.7 | 2.5 | 47.22 | 38.0 | 3 |
| #17 | 800 | 4.99 | 3.7 | 2.7 | 40.84 | 26.6 | 3 |
| #18 | 900 | 5.68 | 5.0 | 2.6 | 70.55 | 38.9 | 2 |

### Example 17

### Pharmacokinetic study. Formulations of ABT-102, 30 or 100 mg Oral Dose in Monkey

Protocol for administration: ABT-102 formulations were administered in a single dose of 30 or 100 mg to groups of six monkeys. The semisolid formulation was melted and administered at a temperature around 37° C. by nasal gavage. The plasma concentrations were determined by HPLC-MS.

Formulation #19 and #20

| | |
|---|---|
| Lipid formulation | 5% ABT-102; 32.3% TPGS; 29.3% Gelucire 44/14; |
| | 15.2% Phosal 53MCT; 18.2% VPD |

TABLE 14
PK parameters for Formulations #19 and #20

| | | | | | | |
|---|---|---|---|---|---|---|
| | ABT-102 | | | | | |
| | dose | Cₘₐₓ | Tₘₐₓ | T_{1/2} | AUC_{0-∞} | |
| Form. | (mg/dog) | (μg/ml) | (hr) | (hr) | (μg · hr/ml) | n |

| | | | | | | |
|---|---|---|---|---|---|---|
| #19 | 30 | 1.03 | 15.0 | 7.7 | 21.0 | 3 |
| #20 | 100 | 1.24 | 8.0 | 4.3 | 22.56 | 3 |

### Example 18

### Pharmacokinetic Study. Formulations of ABT-102, 50 Mg Oral Dose in Dogs—Evaluation of Food Effects

Protocol for administration: formulations were placed in a capsule just prior to dosing. Formulations were administered to histamine-pretreated (fasted) dogs (histamine 30 minutes prior dosing) and food was provided to dogs 30 minutes prior to dosing (non-fasted).

| | |
|---|---|
| Formulation #21 | |
| Semi-solid formulation | 5% ABT-102; 60% Phosal 53 MCT; 10% |
| | PEG 400; 25% Cremophor EL. |
| Formulation #22 | |
| Semi-solid formulation | 6% ABT-102; 59.4% Phosal 53 MCT; 9.9% |
| | PEG 400; 24.7% Tween 20. |

TABLE 15
PK parameters for Formulations #21 and #22

| | ABT-102 dose | Cₘₐₓ | Tₘₐₓ | T_{1/2} | AUC_{0-∞} | |
|---|---|---|---|---|---|---|
| Form. | (mg/dog) | (μg/ml) | (hr) | (hr) | (μg · hr/ml) | n |
| #21 | 50 | 0.20 | 3.0 | 1.6 | 0.69 | 6 |
| #22 | 50 | 0.15 | 3.3 | 11.8 | 0.71 | 6 |
| #21* | 50 | 0.55 | 4.2 | 2.7 | 3.60 | 6 |
| #22* | 50 | 0.45 | 6.3 | 2.8 | 2.88 | 6 |

*food provided 30 minutes prior to dosing

The results indicate a 4-5-fold increase in exposure when administered to non-fasted dogs. The bioavailability of both Formulation #21 and #22 averaged 8% when administered to histamine pretreated (fasted) dogs. Bioavailability of both Formulation #21 and #22 increased to 32%-42% in non-fasted dogs.

### Example 18

### Pharmacokinetic Study. Additional Formulations of ABT-102, 50 mg Oral Dose in Dogs

Each formulation was administered to a group of three histamine pretreated (fasting) dogs; food was returned to the dogs 6 hours after dosing. The 50 mg dose was placed in a capsule just prior to dosing.

| | |
|---|---|
| Formulation #23 | |
| Semi-solid formulation | 6% ABT-102; 61.1% Phosal 53 MCT; 4.7% |
| | PEG 400; 28.2% Labrasol. |
| Formulation #24 | |
| Semi-solid formulation | 6% ABT-102; 51.7% Phosal 53 MCT; 14.1% |
| | PEG 400; 28.2% Labrasol. |
| Formulation #25 | |
| Semi-solid formulation | 5% ABT-102; 52% Phosal 53 MCT; 15% |
| | PEG 400; 28% Labrasol. |
| Formulation #26 | |
| Semi-solid formulation | 6% ABT-102; 56.5% Phosal 53 MCT; 14.5% |
| | PEG 400; 23% Gelucire 44/14. |

TABLE 16
PK parameters for Formulations #23, #25, #25 and #26

| | ABT-102 dose | Cₘₐₓ | Tₘₐₓ | T_{1/2} | AUC_{0-∞} | |
|---|---|---|---|---|---|---|
| Form. | (mg/dog) | (μg/ml) | (hr) | (hr) | (μg · hr/ml) | n |
| #23 | 50 | 0.27 | 2.2 | 1.7 | 1.03 | 3 |
| #24 | 50 | 0.47 | 2.3 | 1.8 | 1.54 | 3 |
| #25 | 50 | 0.32 | 2.7 | 1.4 | 1.04 | 3 |
| #26 | 50 | 0.24 | 3.0 | 1.4 | 0.90 | 2 |

The results from lipid based formulations #23, #24, #25 and #26 resulted in ABT-102 bioavailability values ranging from 10.3 to 16.7%. The best results were obtained with Formulation #24 (6% loading; higher PEG-400), with bioavailability of 16.7%. The bioavailability from the remaining three formulations were all very similar, with values of 13.3%, 12.5% and 10.3% for Formulations #23, #25 and #26, respectively.

### ADDITIONAL EXAMPLES

An accurately weighed quantity of ABT-102 was added into previously labeled 20 ml clear scintillation glass vials. Semisolid excipients were warmed in their original containers over a water bath of approximately 60-70° C. until completely melted prior to weighing. The liquid and melted semisolid excipients were individually weighed into the respective glass vials containing appropriate amount of ABT-102 using disposable pipettes. The vials were sonicated in a warm water bath set at 60° C. until the drug was completely dissolved. For preparation of a solution volume greater than 20 ml, a magnetic stirrer was used to mix the solution maintained at a temperature around 35-50° C. until the drug was completely dissolved.

Dog Studies—Single Dose Formulation Screening

Protocol for Administration (Fasted State)

The details of formulations evaluated for bioavailability at a single dose of 100 mg in beagle dogs are listed in Table 2A. Each formulation was administered in a single dose of 100 mg to a group of three non-histidine pre-treated dogs under fasting conditions. The plasma concentrations were determined by HPLC-MS. The results from this study were compared to those obtained from a 14 mg/kg solution of ABT-102 in PEG-400.

Co-Administration with Food or Ensure

Selected formulations were evaluated for effect of co-administration with food or Ensure Plus on the pharmacokinetics. Administration of Ensure Plus was tried as a potential option to provide a more consistent feeding state. Some formulations were co-administered with 20 ml of a 7.5% aqueous solution of Vitamin E TPGS. Food was administered to the dogs ˜30 minutes prior to dosing. Ensure Plus and Vitamin E TPGS solution were administered to the dogs just prior to dosing.

Method of Dosing Administration

The lipid formulations were administered either by gavage or as hard gelatin capsules filled with the formulation. When the solution was administered by gavage, 3 ml PEG 400 was used to rinse the gavage tubes after administration. Ensure Plus and Vitamin E TPGS was administered by gavage.

TABLE 1A
ABT-102 Formulations evaluated as 100 mg single dose in dogs

| | | | Ad- | Drug | | | | | | |
|---|---|---|---|---|---|---|---|---|---|---|
| Cate- | | Formulation | minis- | Load- | | | AUC ± SEM | | | |
| gory | Lot No. | Composition | tration | ing % | F % ± SEM | | (mcg · hr/mL) | | Cₘₐₓ | Tₘₐₓ |
| Coarse | PEG 400 | ˜14 mg/kg solution in | Gavage | ˜14 | 19.3 ± 0.9 | | 4.87 ± 0.22 | | 1.13 (0.05) | 1.5 (0.0) |
| emul- | solution | PEG 400 | | mg/kg | | | | | | |
| sions | 82106-17 | 1.4% ABT-102, 4.75% | Capsule | 1.40% | 41.3 ± 20.2 | | 7.53 ± 3.31 | | 0.89 (0.28) | 4.7 (0.7) |
| (Oleic | (Pre-DDC | DMSO, 90.25% lipid | | | | | | | | |
| acid | vehicle) | vehicle (OLA:EL:PEG = | | | | | | | | |
| based) | | 81:9:10) | | | | | | | | |
| | 81284-159-1 | 1.5% ABT-102, 75.8% | Capsule | 1.5 | 47.30 ± 11.6 | | 8.16 ± 2.60 | | 1.26 (0.38) | 4.0 (0.0) |
| | | Oleic acid, 8.42% | | | | | | | | |
| | | Cremophor RH40, | | | | | | | | |
| | | 14.28% VP dimer | | | | | | | | |
| | 81284-122-1 | 2% ABT-102, 58.8% | Capsule | 2 | 41.60 ± 5.60 | | 7.41 ± 0.69 | | 1.36 | 4.30 |
| | | Oleic acid, 19.6% | | | | | | | | |
| | | PEG 400, 19.6% | | | | | | | | |
| | | Cremophor RH40 | | | | | | | | |
| | 81284-146- | 2.75% ABT-102, | Capsule | 2.75 | 29.50 ± 2.80 | | 5.44 ± 0.27 | | 1.00 (0.17) | 3.7 (0.3) |
| | 1BB2 | 58.3% Oleic Acid, | | | | | | | | |
| | | 19.65% PEG 400, | | | | | | | | |
| | | 19.2% Cremophor | | | | | | | | |
| | | RH40, 0.1% Vitamin E | | | | | | | | |
| | 81284-130-1 | 5.0% ABT-102, 26% | Capsule | 5 | 3.40 ± 0.50 | | 0.63 ± 0.07 | | 0.21 | 2.50 |
| | | Oleic Acid, 62% PEG | | | | | | | | |
| | | 400, 7% Ethanol | | | | | | | | |
| | 81284-159-2 | 5% ABT-102, 65% | Capsule | 5 | 5.90 | | 1.16 | | 0.23 (0.08) | 3.0 (0.6) |
| | | Oleic Acid, 21.58% | | | | | | | | |
| | | VP dimer, 8.42% | | | | | | | | |
| | | Cremophor RH40 | | | | | | | | |
| | 81284- | 2% ABT-102, 58.8% | Capsule | 2 | 16.40 ± 7.90 | 7.90 | 2.85 ± 1.41 | 1.41 | 0.39 | 0.17 |
| | 122-2 | Capmul MCM, 19.6% | | | | | | | | |
| | | PEG 400, 19.6% | | | | | | | | |
| | | Cremophor RH40 | | | | | | | | |
| | 81284- | 3% ABT-102, 26.2% | Capsule | 3 | 11.60 ± 1.80 | 1.80 | 2.13 ± 0.38 | 0.38 | 0.51 | 2.30 |
| | 122-3 | Capmul MCM, 34.9% | | | | | | | | |
| | | PEG 400, 3.9% Ethanol, | | | | | | | | |
| | | 29.1% Cremophor RH40 | | | | | | | | |
| | 81284- | 5% ABT-102 in Phosal 50 | Capsule | 5 | 16.80 ± 5.50 | 5.50 | 2.98 ± 0.97 | 0.97 | 0.61 | 1.70 |
| | 130-2 | PG:PEG-400:EtOH | | | | | | | | |
| | | (57:28.5:9.5) | | | | | | | | |
| | 81284- | 5% ABT-102 in Phosal 50 | Capsule | 5 | 15.80 ± 3.30 | 3.30 | 2.97 ± 0.69 | 0.69 | .77 (0.19) | 1.5 (0.3) |
| | 146-EE1 | PG:PEG-400:EtOH:Tween 80 | | | | | | | | |
| | | (58:26.2:8.75:2, | | | | | | | | |
| | | by weight) | | | | | | | | |
| | 81284- | 5% ABT-102 in Phosal 50 | Capsule | 5 | 14.40 ± 2.80 | 2.80 | 2.68 ± 0.55 | 0.55 | 1.00 (0.17) | 3.7 (0.3) |
| | 146-1FF1 | PG:PEG-400:EtOH:Tween 80 | | | | | | | | |
| | | (75.0:10.0:8.0:2.0, | | | | | | | | |
| | | by weight) | | | | | | | | |
| | 81284- | 4% ABT-102, 57.6% | Capsule | 4 | 13.70 ± 0.60 | 0.60 | 2.31 ± 0.26 | 0.26 | 0.62 (0.07) | 1.8 (0.2) |
| | 160-3 | Labrasol, 19.2% VP dimer, | | | | | | | | |
| | | 19.2% Vitamin E TPGS | | | | | | | | |
| | 81284- | 5% ABT-102, 65% Oleic | Capsule | 5 | 5.90 | 1.16 | | 0.23 | | 3.0 (0.6) |
| | 159-2 | Acid, 21.58% VP dimer, | | | | | | (0.08) | | |
| | | 8.42% Cremophor RH40 | | | | | | | | |
| | 81284- | 3.39% ABT-102 in 75% | Capsule | 3.39 | 34.30 ± 1.60 | | 6.88 ± 0.67 | | 3.7 (0.0) | 1.46 (0.02) |
| | 154-13 | Gelucire 44/14: | | | | | | | | |
| | | 25% Cremophor RH40 | | | | | | | | |
| | 81284- | 3% ABT-102, 67.9% | Capsule | 3 | 38.00 ± 4.80 | | 7.29 ± 0.76 | | 1.62 (0.18) | 2.3 (0.3) |
| | 160-1 | Vitamin E TPropylene | | | | | | | | |
| | | glycolS, 14.55% | | | | | | | | |
| | | Propylene glycol, 14.55% | | | | | | | | |
| | | VP Dimer | | | | | | | | |
| | 81284- | 4% ABT-102, 67.2% | Capsule | 4 | 62.5 ± 36.80 | | 11.79 ± 7.01 | | 1.81 (0.75) | 2.5 (0.8) |
| | 160-2 | Vitamin E TPGS, 14.4% | | | | | | | | |
| | | PG, 14.4% VP Dimer | | | | | | | | |
| | 81284- | 4% ABT-102, 67.2% | Capsule | 4 | 28.10 ± 3.00 | | 4.68 ± 0.53 | | 1.26 (0.05) | 3.0 (0.0) |
| | 174-3 | Vitamin E TPGS, 14.4% | with 25 | | | | | | | |
| | | PG, 14.4% VP Dimer | ml of | | | | | | | |
| | | | TPGS | | | | | | | |
| | | | 7.5% | | | | | | | |
| | | | aqueous | | | | | | | |
| | | | solution | | | | | | | |
| | | | predose | | | | | | | |
| | 81284- | 4% ABT-102, 62.4% | Capsule | 4 | 39.40 ± 4.10 | | 7.57 ± 0.95 | | 1.25 (0.21) | 3.0 (0.0) |
| | 174-2 | Vitamin E TPGS, 9.2% | with 25 | | | | | | | |
| | | Gelucire 44/14, 14.4% VP | ml of | | | | | | | |
| | | Dimer | Vitamin | | | | | | | |
| | | | E TPGS | | | | | | | |
| | | | 7.5% | | | | | | | |
| | | | aqueous | | | | | | | |
| | | | solution | | | | | | | |
| | | | predose | | | | | | | |
| | 81396- | 4% ABT-102, 62.4% | Capsule | 4 | 25.50 ± 2.60 | | 4.21 ± 0.20 | | 1.13 (0.07) | 2.2 (0.4) |
| | 051-1 | Vitamin E TPGS, 19.2% | | | | | | | | |
| | | Gelucire 44/14, 14.4% VP | | | | | | | | |
| | | Dimer | | | | | | | | |
| | 81396- | 4% ABT-102, 58.2% | Capsule | 4 | 47.30 ± 8.00 | | 8.84 ± 1.69 | | 1.85 (0.32) | 3.3 (0.3) |
| | 051-2 | Vitamin E TPGS, 28.8% | with 25 | | | | | | | |
| | | Gelucire 44/14, 14.4% VP | ml of | | | | | | | |
| | | Dimer | Vitamin | | | | | | | |
| | | | E TPGS | | | | | | | |
| | | | 7.5% | | | | | | | |
| | | | aqueous | | | | | | | |
| | | | solution | | | | | | | |
| | | | predose | | | | | | | |

Single Dose Studies in Dogs—Formulation Screening for Total Exposure Protocol for Administration

The details of formulations screened for achieving desired total exposure by administering higher doses in beagle dogs are listed in Table 3A.

TABLE 2A
ABT-102 Formulations evaluated in higher doses for total exposure

| | | Drug | | AUC | | Dose | |
|---|---|---|---|---|---|---|---|
| Lot No. | Formulation Composition | Loading % | BA % | (mcg · hr/mL) | SEM | (mg) | Administration |
| 81283-4-1 | 4% ABT-102, 52.8% | 4 | 90.7 | 84.3 | 24.8 | 480 | Predosed with 25 ml |
| | Vitamin E TPGS, 28.8% | | | | | | 10% Vitamin E |
| | Gelucire 44/14, 14.4% | | | | | | TPGS aqueous |
| | VP Dimer | | | | | | solution |
| 81283-14-3 | 4% ABT-102; 52.8% | 4 | 41.2 | 38.6 | 11.0 | 480 | Predosed with 30 mL |
| | Vitamin E TPGS, 28.8% | | | | | | EnsurePlus |
| | Gelucire 44/14, 14.4% | | | | | | |
| | VP Dimer | | | | | | |
| 81283-14-4 | 4% ABT-102; 52.8% | 4 | 46.2 | 42.1 | 6.0 | 480 | No predose |
| | Vitamin E TPGS, 28.8% | | | | | | |
| | Gelucire 44/14, 14.4% | | | | | | |
| | VP Dimer | | | | | | |
| 81283-18-1 | 4% ABT-102, 52.8% | 4 | 38.0 | 47.2 | 8.4 | 640 | No predose, food |
| | Vitamin E TPGS, 28.8% | | | | | | after 12 hr |
| | Gelucire 44/14, 14.4% | | | | | | |
| | VP Dimer | | | | | | |
| 81283-18-2 | 5% ABT-102, 44% | 5 | 26.6 | 40.8 | 4.0 | 800 | No predose, food |
| | Vitamin E TPGS, 36% | | | | | | after 12 hr |
| | Gelucire 44/14, 15% VP | | | | | | |
| | Dimer | | | | | | |
| 81283-18-3 | 5% ABT-102, 44% | 5 | 40.0 | 60.9 | 1.5 | 800 | Co-dosed with 4 ml |
| | Vitamin E TPGS, 36% | | | | | | 37.5% Vitamin E |
| | Gelucire 44/14, 15% VP | | | | | | TPGS in capsule, |
| | Dimer | | | | | | food after 12 hr |
| 81283-22-1 | 5% ABT-102, 44% | 5 | 53.0 | 87.3 | 19.2 | 900 | 4 ml 37.5% Vitamin |
| | Vitamin E TPGS, 36% | | | | | | E TPGS in capsule, |
| | Gelucire 44/14, 15% VP | | | | | | food after 12 hr |
| | Dimer | | | | | | |
| 81283-14-2 | 6% ABT-102; 32% | 6 | 38.7 | 35.6 | 1.8 | 480 | Predosed with 25 ml |
| | Vitamin E TPGS, 29% | | | | | | 10% Vitamin E |
| | Gelucire 44/14, 15% | | | | | | TPGS aqueous |
| | Phosal, 18% VP Dimer | | | | | | solution |
| 81283-22-2 | 6% ABT-102; 43% | 6 | 37.4 | 61.5 | 10.3 | 900 | Co-dosed with 4 ml |
| | Vitamin E TPGS, 36% | | | | | | 37.5% Vitamin E |
| | Gelucire 44/14, 15% VP | | | | | | TPGS in capsule, |
| | Dimer | | | | | | food after 12 hr |
| 81283-14-1 | 8% ABT-102; 25% Vitamin E | 8 | 42.3 | 39.6 | 8.7 | 480 | Predosed with |
| | TPGS, 32% Gelucire 44/14, | | | | | | 25 ml 10% |
| | 16% Phosal, 19% VP Dimer | | | | | | TPGS aqueous |
| | | | | | | | solution |
| 81283-18-4 | 8% ABT-102; 25% Vitamin E | 8 | 38.9 | 70.6 | 5.5 | 900 | Co-dosed with |
| | TPGS, 32% Gelucire 44/14, | | | | | | 4 ml 37.5% |
| | 16% Phosal, 19% VP Dimer | | | | | | TPGS in |
| | | | | | | | capsule, food |
| | | | | | | | after 12 hr |
| 81283-22-4 | 8% ABT-102; 35% Vitamin E | 8 | 42.2 | 67.1 | 8.0 | 900 | 4 ml 37.5% |
| | TPGS, 35% Gelucire 44/14, | | | | | | TPGS in |
| | 22% VP Dimer | | | | | | capsule, food |
| | | | | | | | after 12 hr |
| 81283-22-3 | 8% ABT-102; 35% Vitamin E | 8 | 31.5 | 52.8 | 2.9 | 900 | No |
| | TPGS, 35% Gelucire 44/14, | | | | | | predose, food |
| | 22% VP Dimer | | | | | | after 12 hr |
| 81283-25 | 8% ABT-102; 25% Vitamin E | 8 | 21.3 | 48.0 | 15.4 | 1200 | No predose |
| | TPGS; 32% Gelucire 44/14; | | | | | | |
| | 16% Phosal; 19% VP Dimer | | | | | | |
| 81283-25 | 8% ABT-102; 25% Vitamin E | 8 | 78.9 | 169 | 21.3 | 1200 | No predose, |
| | TPGS; 32% Gelucire 44/14; | | | | | | fed |
| | 16% Phosal; 19% VP Dimer | | | | | | |
| 81283-30 | 8% ABT-102, 35% Vitamin E | 8 | 38.1 | 62.4 | 7.8 | 900 | No predose, |
| | TPGS, 35% Gelucire 44/14, | | | | | | food after 6 hr |
| | 22% VP dimer | | | | | | |
| 81283-30 | 8% ABT-102, 35% Vitamin E | 8 | 27.7 | 45.9 | 5.2 | 900 | 4 ml 37.5% |
| | TPGS, 35% Gelucire 44/14, | | | | | | TPGS in |
| | 22% VP dimer | | | | | | capsule; food |
| | | | | | | | after 6 hr |
| 81283-30 | 8% ABT-102, 35% Vitamin E | 8 | 78.8 | 130.4 | 21.1 | 900 | No predose, |
| | TPGS, 35% Gelucire 44/14, | | | | | | food 0.3 hr |
| | 22% VP dimer | | | | | | prior to dose |
| 81283-30 | 8% ABT-102, 35% Vitamin E | 8 | 70.7 | 110.9 | 26.5 | 900 | Predosed with |
| | TPGS, 35% Gelucire 44/14, | | | | | | 4 ml 37.5% |
| | 22% VP dimer | | | | | | TPGS in |
| | | | | | | | capsule; food |
| | | | | | | | 0.3 hr prior to |
| | | | | | | | dose |

Fasted State:

Formulations were administered in increasing doses of 480 mg, 640 mg, 800 mg, 900 mg and 1000 mg to groups of three to six dogs under fasting conditions. The lipid formulations were administered as hard gelatin capsules filled with the formulation. The plasma concentrations were determined by HPLC-MS.

Co-Administration

Selected formulations were evaluated for effect of co-administration with food or Ensure Plus on the pharmacokinetics. Administration of Ensure Plus was tried as a potential option to provide a more consistent feeding state. Some formulations were co-administered with a capsule filled with 4 ml of a 37.5% solution of Vitamin E TPGS in PEG 400. Food was administered to the dogs at different times before and after dosing. Ensure Plus and Vitamin E TPGS solution were administered to the dogs just prior to dosing. Vitamin E TPGS solution was administered to the dogs as a 37.5% solution in PEG 400 filled in a hard gelatin capsule. Ensure Plus was administered by gavage.

Single Dose Studies in Dogs—Evaluation of Dose Escalation rResponse

Protocol for Administration

The details of formulations evaluated for the effect of dose on ABT-102 plasma concentrations following a single oral dose administration in dogs are listed in Table 4. Formulations were evaluated for the effect of dose on the ABT-102 plasma concentrations following single dose oral administration in dogs. Three separate studies were conducted, each covering a dose range of 100 mg, 300 mg, 600 mg and 900 mg. Two of these studies used a formula of 8% ABT-102, 35% Vitamin E TPGS, 35% Gelucire 44/14, 22% VP dimer, The capsules were administered to dogs in the fasted state, in one study food was provided to the dogs ˜6 hours after dosing, and in the other study, a 4 ml 37.5% Vitamin E TPGS in capsule was co-dosed and food was provided to the dogs 6 hr after dosing. The third study evaluated a formula with slightly lower drug loading (6.5% ABT-102, 37.4% Vitamin E TPGS, 37.4% Gelucire 44/14, 18.7% VP dimer). This formula contains the maximum amount of excipient that is accommondated by three gelatin capsules. In all studies, the 900 mg formulation was diluted with the vehicle to obtain lower doses of 100, 300 and 600 mg in order to maintain the quantity of excipients roughly equivalent and the number of capsules equal. The plasma concentrations were determined by HPLC-MS.

TABLE 3A
ABT-102 Formulations evaluated for dose response in dogs

| Experiment | | Drug | | | | Dose | |
|---|---|---|---|---|---|---|---|
| Lot # | Formulation Composition | Loading | BA % | AUC | SEM | (mg) | Predosing |
| 81283-36 | 8% ABT-102, 35% Vitamin E | 8 | 170 | 31 | 5.3 | 100 | No Vitamin E TPGS, |
| | TPGS, 35% Gelucire 44/14, | | | | | | food after 6 hr |
| | 22% VP dimer | | | | | | |
| 81283-36 | 8% ABT-102, 35% Vitamin E | 8 | 59.3 | 32.5 | 3.7 | 300 | No Vitamin E TPGS, |
| | TPGS, 35% Gelucire 44/14, | | | | | | food after 6 hr |
| | 22% VP dimer | | | | | | |
| 81283-36 | 8% ABT-102, 35% Vitamin E | 8 | 34.2 | 37.8 | 3.4 | 600 | No Vitamin E TPGS, |
| | TPGS, 35% Gelucire 44/14, | | | | | | food after 6 hr |
| | 22% VP dimer | | | | | | |
| 81283-36 | 8% ABT-102, 35% Vitamin E | 8 | 31.4 | 48.5 | 7.2 | 900 | No Vitamin E TPGS, |
| | TPGS, 35% Gelucire 44/14, | | | | | | food after 6 hr |
| | 22% VP dimer | | | | | | |
| 81283-40 | 8% ABT-102, 35% Vitamin E | 8 | 129.3 | 23.6 | 4.6 | 100 | 4 ml 37.5% Vitamin E |
| | TPGS, 35% Gelucire 44/14, | | | | | | TPGS in capsule; food |
| | 22% VP dimer | | | | | | after 6 hr |
| 81283-40 | 8% ABT-102, 35% Vitamin E | 8 | 53.8 | 29.8 | 4.8 | 300 | 4 ml 37.5% Vitamin E |
| | TPGS, 35% Gelucire 44/14, | | | | | | TPGS in capsule; food |
| | 22% VP dimer | | | | | | after 6 hr |
| 81283-40 | 8% ABT-102, 35% Vitamin E | 8 | 32.2 | 35.6 | 5.3 | 600 | 4 ml 37.5% Vitamin E |
| | TPGS, 35% Gelucire 44/14, | | | | | | TPGS in capsule; food |
| | 22% VP dimer | | | | | | after 6 hr |
| 81283-40 | 8% ABT-102, 35% Vitamin E | 8 | 34.5 | 55.9 | 3.1 | 900 | 4 ml 37.5% Vitamin E |
| | TPGS, 35% Gelucire 44/14, | | | | | | TPGS in capsule; food |
| | 22% VP dimer | | | | | | after 6 hr |
| 81283-45-1 | 6.5% ABT-102, 37.4% Vitamin | 6.5 | 128.6 | 23.3 | 0.4 | 100 | No Vitamin E TPGS, |
| | E TPGS, 37.4% Gelucire 44/14, | | | | | | food after 6 hr |
| | 18.7% VP dimer | | | | | | |
| 81283-45-1 | 6.5% ABT-102, 37.4% Vitamin | 6.5 | 33.4 | 36.8 | 7.1 | 600 | No Vitamin E TPGS, |
| | E TPGS, 37.4% Gelucire 44/14, | | | | | | food after 6 hr |
| | 18.7% VP dimer | | | | | | |
| 81283-45-1 | 6.5% ABT-102, 37.4% Vitamin | 6.5 | 30.1 | 51.8 (n = 1) | | 900 | No Vitamin E TPGS, |
| | E TPGS, 37.4% Gelucire 44/14, | | | | | | food after 6 hr |
| | 18.7% VP dimer | | | | | | |

Dog Studies—Multiple Dose Studies (Protocol for Administration)

The details of formulations used for multiple dose studies in beagle dogs are listed in Table 5. Groups of four dogs (2 male, 2 female per group) received an oral dose of either the drug-containing lipid formulation or the placebo formulation at a dose of 10 mg or 60 mg/kg once daily, for 2 weeks. The formulation composition was adjusted to administer roughly equivalent amounts of Vitamin E TPGS, Gelucire 44/14 and VP dimmer to all the dogs. Each dog received 3 capsules once daily, for 2 weeks, administered under fasting conditions. Food was provided to the dogs ˜6 hours after dosing. Plasma samples were obtained from each dog on Day 0, Day 5 and Day 15. Plasma concentrations of parent drug and two metabolites (A-892856 (hydroxyl metabolite) and A-892667 (carboxylic acid metabolite) were determined by HPLC/MS at the completion of the two-week dosing interval.

TABLE 4A
ABT-102 formulations evaluated for multiple dose studies in dogs

| | | mg/dose | Drug |
|---|---|---|---|
| Experiment Lot # | Formulation Composition | of 11.25 g | Loading |
| 81283-49A and | 8% ABT-102, 35% Vitamin E | 900 | 8% |
| 81283-51A | TPGS, 35% Gelucire 44/14, | | |
| | 22% VP dimer | | |
| 81283-49B and | 5.3% ABT-102, 36% Vitamin E | 600 | 5.30% |
| 81283-51B | TPGS, 36% Gelucire | | |
| | 44/14, 22.6% VP dimer | | |
| 81283-49C and | 0.9% ABT-102, 37.7% | 100 | 0.90% |
| 81283-51C | Vitamin E TPGS, 37.7% | | |
| | Gelucire 44/14, 23.7% VP dimer | | |
| Placebo | 38% Vitamin E TPGS, | N/A | N/A |
| | 38% Gelucire 44/14, 23.9% | | |
| | VP dimer | | |

Monkey Studies—Single Dose and Dose Response
Protocol for Administration

The details of formulations screened in monkeys for single dose and dose response studies in cynomologous monkeys are listed in Table 6. Each formulation was administered in a single dose of 100 mg to a group of three monkeys. The plasma concentrations were determined by HPLC-MS

Method of Administration

The lipid formulations were administered either by gavage.

Monkey Studies—Multiple Dose Studies

Administration Protocol

Each formulation was administered in a single dose of 100 mg to a group of three monkeys under fasting conditions. The plasma concentrations were determined by HPLC-MS The lipid formulations were administered by nasal gavage. Semisolid formulations were warmed at a temperature of 50° C.±5° C. until the material reached a liquid state prior to dosing. The formulation was then maintained in a liquid state until administered to the animals at a temperature of 37° C.±5° C.

Rat Studies—Single Dose and Dose Response Studies

Protocol for Administration

Each formulation was administered at a maximum of 3 ml/kg to a group of three rats. The rats were permitted food (normal diet) and water ad libitum. Blood samples were obtained from each rat for 24 hours after dosing. The plasma concentrations were determined by HPLC-MS.

Dog Studies—Single Dose Formulation Screening

The desired goal was a bioavailability of 40% (with variability less than 30%) in fasted dogs using a single dose of 100 mg. It was also desirable to obtain a drug loading of at least 5% in order to ensure that the volume needed for higher doses would not exceed excipient limits that could be administered. During the pre-DDC formulation screening, a lipid formulation consisting of 90.25% of a lipid vehicle (with a composition of oleic acid: cremophor EL:PEG-400 at 81:9:10 weight ratio), 4.75% DMSO and 1.4% ABT-102 was used. Although a bioavailability of 41.3% was achieved using this formula, the need of using DMSO to dissolve the API was not desirable for a toxicological evaluation purpose.

Based on the data obtained for the pre-DDC lipid formulation, a series of oleic acid-based lipid formulations were developed using Cremophor RH40 as a surfactant, and either PEG-400 or VP dimer as cosolvents, with a drug loading ranging from 1.5% to 5%. Additionally formulations based on a medium chain mono- and diglycerides, Capmul MCM, were also made covering a drug loading range from 2-3%. These formulations yielded coarse emulsions upon dispersing at a 1:100 (w/v) ratio in 0.1N HCl or water. The results of evaluation of the Oleic acid-based and Capmul MCM-based formulations in dogs is presented in Table 2. The bioavailability in dogs as a function of drug loading is also plotted in FIG. 2. The Capmul-based formulations showed a bioavailability of 11.6% to 16.4% at a low drug loading of 3% and 2%, respectively. The oleic acid-based formulations provided bioavailability ranging from 3.4% (at high drug loading of 5%) to 47.3% (at low drug loading of 1.5%).

For both types of formulations, the bioavailability exhibited a strong dependence on drug loading, with the oleic acid-based formulation providing relatively higher bioavailability than the capmul-based formulation at the same drug loading level. Since ABT-102 exhibits very poor aqueous solubility and its solubility in oleic acid and capmul is also limited, the capacity of these lipid systems to retain the drug in a solubilized state is reduced as the drug loading is increased. Once suspended in water, the drug is likely to precipitate out and this may be the reason of reduced bioavailability at higher drug loading levels.

Next, lipid formulations were prepared using Phosal 50 PG that would lead to more finely dispersed systems. First, a formula containing 5% ABT-102 in a lipid vehicle consisting of phosal 50 PG: PEG-400: EtOH at 57: 28.5: 9.5 weight ratio was tested in dogs. This formula gave a bioavailability of 16.8% in dogs. Polysorbate 80 was then incorporated as a surfactant to further enable formation of a finely dispersed system. In spite of forming a more uniformly dispersed emulsion upon dispersing in aqueous medium, no significant differences in in vivo absorption were seen in the formulations as a result of addition of surfactant (lots 81284-146-EE1 and 81284-146-FF1). Overall, the Phosal-based formulations showed higher bioavailability for a 5% drug loading as compared with the oleic acid-based formulations and the capmul MCM based-formulations. However, the bioavailability achieved was much lower than the desired target at this level of drug loading.

A number of labrasol-based finely dispersed formulations were provided by PARD LU. These formulations yielded bioavailability values ranging from 2%-23.3% for drug loading ranging from 4-6%. The bioavailability as a function of drug loading follows the same trend as the phosal-based formulations. This again shows that the in vivo absorption could be related to the extent of dispersion of oil droplets. One interesting observation is that when transcutol CG/Capmul MCM/propylene glycol in lot 81284-154-24 are replaced by VP dimer and Vit. E TPGS in lot 81284-167-1, at similar drug loading (˜6%), the dog bioavailability is increased from 2% to 11%. This deviation from the trend shows that Vit. E TPGS as a surfactant may further enhance the dispersibility of the system.

Following this logic and guided by in vitro dispersibility tests, a number of Self Emulsifying Drug Delivery systems-(SEDDS)based formulations were developed using a comibination of surfactant such as Cremophor RH40, Gelucire 44/14 and Vitamin E TPGS, and solvents such as propylene glycol and VP dimer. Upon dispersing in 0.1N HCl solution, the dispersions obtained with these systems were colloidal translucent solutions. Again, these formulation yielded bioavailability in a manner that follows the BA-drug loading trend as showed by the finely dispersed lipid systems discussed above.

In order to further increase the bioavailability and decrease the dependence on drug loading, two approaches were taken. First, the formulations were predosed with an aqueous solution of Vit. E TPGS (25 ml of a 7.5% solution). Secondly, Gelucire 44/14 was included in the formulation in increasing levels. The predosing approach was chosen upon observation that dispersing the TPGS-based lipid formulations in the a TPGS solution gave a clear solution whereas when dispersed in water or 0.1N HCl, a translucent solution was resulted. Based on the high tolerability of TPGS in animal species (numbers?), a predosing of 25 ml of a 7.5% aqueous solution of TPGS (1.875 g) administered by gavage prior to dosing the TPGS-based formulation would be feasible for the purpose of this study. The results were very encouraging. The same 4% formulation that yeilded 25.5% bioavailability without predosing (lot 81396-051-1) would now gave a bioavailability of 39.4% (lot 81284-174-2). In addition to TPGS predosing, increasing Gelucire 44/14 in the formulation also resulted in a significant improvement in the bioavailability (from 24.8% for lot 81396-051-3 to 47.8% for lot 81396-051-2). For these formulations, the predosing also eliminated the dependence of bioavailability on drug loading. The high Gelucire level coupled with predosing of TPGS solution enabled a drug loading as high as 8% to achieve a bioavailability above 40% (lot 81283-14-1).

Hence for a single dose of 100 mg, the desired goal of 40% BA with a DL of NLT 5% was achieved. SEDDS systems performed best among all lipid systems. Predosing with a TPGS solution further enhanced BA and minimized drug loading effect

Increased Dose

The desired target was an exposure level of AUC of 50-60 μg●hr/ml. Ideally this had to be administered in 3 capsules, up to 4 was acceptable. Excipient quantity to be maintained within acceptable safety limits and predosing with TPGS solution was to be avoided.

Dog Studies—Formulation Screening for Total Exposure

Formulations and doses experimented to achieve this are listed in Table 3. The doses were increased from 100 mg up to 1200 mg with some minor variations in the formulations. The dose was maintained in 3-4 capsules and drug loading was increased up to 8%. The excipient quantity up to 1200 mg fulfilled the safety requirements. Predosing was changed to a capsule filled with TPGS solution in PEG/PG. Higher doses were administered without predosing. AUC/dose exhibit linear relationship up to 900 mg and emesis in dogs was observed at high dose (>900 mg); Predosing TPGS at high doses does not further enhance AUC.

Dog Studies—Dose Escalation/Dose Response Studies

Two formulations were selected for dose response studies. The dose response was adequate.

Dog Studies—Multiple Dose Studies

The formulation selected for multiple dose studies in dogs was based on the exposure obtained, safety of excipients and dose response seen. However, after the multiple dose studies, it was found that plasma concentration declined dramatically with multiple dosing, possibly due to induction of metabolism. The end-study samples were analyzed and found to be still stable.

Monkey Studies—Single Dose and Dose Response

The formulations were evaluated in an alternative species, the cynomlogous monkey. Since the formulations have to be administered by nasal gavage, the formulation was modified to be more liquid at 37 C. For this Phosal 50 PG and Phosal 53 MCT were included in the formulation. This formulation was liquid at 37° C., although it slowly becomes a semisolid upon cooling to room temperature.

Based on screening work from R4P3, two families of carrier solutions were investigated: one using Phosal 53 MCT (American Lecithin Company, Oxford, Conn.) as primary solvent and the other using oleic acid (Mednique 6322, Cognis corporation, Florence, Ky.) as primary solvent. In both cases, PEG 400 (Lutrol 400 NF or Pluracare E400 or from BASF corp., Mount Olive, N.J.) was used as a drug solubility enhancer. Emulsifiers used were Polysorbate 80 (Crillet 4HP, Croda Inc., Parsippany, N.J.) and Polyoxyl 35 castor oil (Cremophor EL, BASF corp., Mount Olive, N.J.). Antioxidants studied were: Butylated Hydroxytoluene (Abbott code 04703KJ00), Citric acid (Sigma Aldrich co., Inc. Milwaukee, Wis.), L-Ascorbic acid (Sigma Aldrich co., Inc. Milwaukee, Wis.), L-Ascorbic 6-palmitate (Sigma Aldrich co., Inc. Milwaukee, Wis.) and dl-alpha tocopherol (Sigma Aldrich co., Inc. Milwaukee, Wis.). The main drug solubilizers in Phosal 53 MCT are lecithin (phosphadidylcholine) and medium chain triglyceride oil. The complete composition of Phosal 53 MCT is given in Table 5. Table 5 also lists information on the functions of the components, along with their compendial status. Although Phosal 53 MCT is not an approved excipient, all of its components are used in numerous pharmaceutical, cosmetic and nutritional applications.

### Drug Solubility Determination

Approximately 100-400 mg of compound was weighed into a 4 ml glass vial, to which 2 ml of Blend was added. The vials were then vortexed and sonicated for 10 minutes. After wrapping the vials with aluminum foils to protect the API from light-induced degradation, they were placed in a water bath held at 25° C. and agitated for 2 days.

Once the samples were filtered and diluted, 100 μl of solute was pipeted into a 25 ml volumetric flask for HPLC analysis. Once the exact weight was recorded, the sample dissolved in methanol. The exact weight dilution was 25× (40 μl of sample/960 μl of mobile phase).

### Preparation of Drug Solutions for Formulation Studies

Carrier solutions were prepared first by weighing individual excipients into an amber bottle or vial. Mixtures of liquid ingredients were homogenized by vortexing followed by sonication. The API was then added to the carrier solution in a second amber bottle. The API dissolution process was aided by vortexing, followed by 20 to 30 minutes of sonication until a clear liquid was obtained. The solution would then be stored overnight at room temperature before use.

In some cases, the solutions were prepared under a nitrogen atmosphere, using a 280 liter glove bag (Aldrich AtmosBag, model Z11282-8, Aldrich Chemical Company, Milwaukee, Wis.) with a containment box. Once all necessary equipment was placed inside the glove bag, purging was achieved by first pushing air out, and then by inflating the bag with nitrogen. The bag was then compressed again, sealed, re-inflated with nitrogen and a positive pressure was maintained throughout the manufacturing process. Nitrogen purity was 99.995%.

### Pharmacokinetics Studies in Dog

The dog PK work was performed under fasted conditions. Plasma concentration of parent drug were determined by HPLC-MS.

API solutions were administered to dogs either orally in soft gel capsules or by gavage after dilution in apple juice. The soft gel capsules used were hydrophilic, air-filled, capsules (L3DXHB, Cardinal Health, Inc. Dublin, Ohio). The gelatin capsule were filled with a syringe (Gage 20 needle) and heat-sealed with a spatula. In the case of apple juice dilution studies, the API solutions and apple juice were supplied separately and mixed immediately before administration. Apple juice for dilution studies was obtained from 1.89 bottles purchases at Dominick's under the label “100% Apple Juice with Added Vitamin C”.

### Stability Studies

Two separate stability studies were undertaken. The first study was to establish the stability of one Phosal 53 MCT formulation (F11 with 2.5% w/w drug) and one oleic acid based formulation (F13 with 2.5% w/w drug) in 1 cc syringes and type III amber bottles held at 5° C., 25° C./60% RH and 40° C./75% RH for at least one month. The 1 cc syringes and amber bottles used, as well as the rationale for their selection are described at the end of this section. All samples used for the first stability study were prepared in air. Drug lot #1251524-0 was used to prepare the solutions that went into the first stability study.

The objective of the second stability study was to establish the effectiveness of antioxidants added to oleic acid based formulations. All samples were prepared under a nitrogen blanket. To facilitate visual observation of color changes and phase separation, the containers were clear scintillation vials. During storage, the vials were covered with aluminum foils for protection from light. Storage conditions were 5° C., 25° C./60% RH and 40° C./75% RH. A total of 10 formulations were studied. As in the first stability study, the drug loading was 2.5% w/w in all cases. Drug lot# lot 16-632-AL was used to prepare the solutions that went into the second stability study.

Cold room LC943137 located in NC-R14 was used for storage at 5° C. Chambers LC932330 and LC932329 located in NC-R13-142 were used for storage at 25° C./60% RH and 40° C./75% RH, respectively. The bottles and syringes selected for stability studies are listed below:

Bottles and Closures:

10 cc type III amber, special part #WO12442 (Alcan Packaging PPC Inc, Millville, N.J.)
20-400 cap with Teflon faced foamed PE liner, Cat #239229 (drawing #A=WO10638) (Alcan Packaging PPC Inc, Millville, N.J.)
   Syringes and Syringe Caps:
Baxa 1 cc syringe with caps, item 7101 (Baxa corporation, Englewood, Co)
HSW Norm-Ject 1 cc syringe, item A1 (Air-Tite Co. Inc., Virginia Beach, Va.) with item BUCC clear caps

The amber bottles are Abbott commodity items. Since larger bottles will be needed for manufacturing and shipment, we verified that the composition of the 10 cc bottles is identical to that of the larger type III amber bottles from the same supplier.

Both syringes feature pistons and barrels made of high molecular weight polyolefins that are compatible with most pharmaceutical ingredients. In the Baxa syringes, the clearance between the barrel and the piston is sealed with small silicon rings and friction is reduced with a coating of medical grade silicone oil. By contrast, the HSW syringes are gasket free, thanks to a precision molding process. They are also lubricant free, thanks to the smooth finish of the 2 components. The absence of elastomer and lubricant in the HSW design greatly reduces the risk of product contamination.

### Potency Assays

Potency Assays in Support of the Stability Studies were Carried Out by PARD Analytical.

### Drug Solubility and Dog Pharmacokinetics

With density varying from one vehicle formulation to another, it was found more practical to formulate on a weight % basis than on a weight per unit volume basis. Once the final formulation is selected, the density of the vehicle will be measured and concentrations will be reported in mg/ml for patient dosing on a volumetric basis. Except in the case of F13, all dog PK studies reported herein delivered a drug dose of 100 mg.

Phosal 53 MCT-Based Formulations

The solubility and dog PK data for 5 formulations studied are summarized in Table 6B. The dog PK results are summarized in both Table 6B. All drug solubility values were determined at room temperature.

The reference carrier formulation (abbreviated as “baseline” in Table 6B) screened by pre-formulation (R4P3) had 9.7% w/w drug solubility and a bioavailability in dog of 37.4%. In the early stage of the formulation effort, the objective was to achieve a maximum drug loading of 100 mg/ml, a saturated solubility at least 50% greater than the maximum drug loading, and, if possible, to improve upon the bioavailability of the R4P3 prototype. When the 10% w/w the ethanol present in the baseline formulation were substituted with 10% w/w PEG 400 (F11-4), drug solubility was raised from 9.7% w/w to 13.9% w/w, i.e., a significant improvement, but not enough to meet the saturated solubility goal of 15% w/w. The trend for bioavailability was also downward from the baseline formulation to F11-4. Increasing the level of PEG 400 would have probably increased drug solubility with an additional bioavailability penalty and an increased stability risk. Rather, a consensus was reached to relax the maximum drug loading requirement to 7.5% w/w and to increase dog bioavailability while maintaining saturated solubility above 150% of the maximum drug loading.

Carrier F11-4, with a drug loading of 7.5% achieved all the above objectives. However, F11-4 was too viscous to be easily pulled into a syringe at room temperature in a clinic setting. To address this challenge, vehicle F11-5 with 5% w/w ethanol was introduced. The lower Phosal 53 MCT content reduced both the drug solubility and bioavailability. Although the resulting drug solubility of 12.2% w/w was above 150% of the maximum drug loading, additional work was focused on improving bioavailability while maintaining the drug solubility target. To achieve this, carrier F11-5 was modified by introducing 0.5% Polysorbate 80 to improve emulsification. The properties of the resulting solution (F11-6) are shown in Table 6. With F11-6, drug solubility was maintained, while viscosity was lowered to an acceptable level for handling at the clinics. In addition, bioavailability was increased significantly compared to that of earlier formulations.

F11-6 was the lead Phosal 53 MCT carrier formulation when the project was transferred to LU. In the event that impurities from PEG 400 might later be found to be a cause of API degradation, a PEG-free vehicle was developed. An example of such a PEG free carrier is F11-7. Although API solubility in this carrier was not measured, its composition is so close to that of R4P3 prototype “Baseline” (only 0.5% Polysorbate added), that it likely to be in the 9 to 10% w/w range. As a result F11-7 would not be able to sustain as high a drug loading as F11-6. The dog PK data summarized in Table 6 indicates that its bioavailability in dog is high and very close to that of F11-6.

Earlier toxicity studies of Phosal 53 MCT performed in dog by R4EK did not show any evidence of dose non-linearity. Therefore, the variations in drug loading seen across Table 6B are not thought to affect dog bioavailability.

Effect of Dilution Studies in Apple Juice:

The effect of 1:20 w/w dilution in apple juice on the bioavailability in dog of carriers “baseline”, F11-6 and F11-7 with 5% w/w drug loading was also studied. The dose was 100 mg in all cases, but the drug loading varied. All data is summarized in Table 7B.

The baseline carrier with 5% drug loading and diluted in apple juice was compared with historical data generated with in the same group of dogs, but administered with soft gel capsules and with a drug loading of 6.5% w/w. The same was done with F11-6 carrier with 5% drug, except that the control was obtained with a drug loading of 7.5% w/w in soft gel capsules. The plasma profiles for these two formulations are shown herein. Within the variability of the small number of dogs, there was no significant difference in pharmacokinetics between diluted and undiluted F11-6, or between diluted and undiluted baseline formulation.

A similar dilution study was performed on carrier F11-7 with 5% w/w drug. Soft gel capsule dosing was performed in another group of dogs, but drug loading remained the same. Plasma concentrations declined slightly after dilution in apple juice. The variability of diluted F11-7 was actually lower than that seen from the administrations of capsules. For comparison, the baseline and F11-6 formulations are shown on the same plot. The lowest bioavailability was obtained with the baseline, with bioavailability increasing through the addition of either Polysorbate 80 or PEG/Polysorbate 80.

Visual observations were also made on the stability of the emulsions obtained by diluting the above formulations in apple juice. The stability of the emulsions ranked as followed: F11-7 >>F11-6>baseline. Polysorbate 80 had a significant impact on the long-term stability of the emulsions, particularly in the case of F11-7.

Oleic Acid-Based Formulations

The solubility and dog PK data for 6 formulations studied are summarized in Table 8 B. The dog PK results are summarized in Table 8.

The oleic acid formulations are similar to those used for Norvir and Kaletra. These formulations were considered as alternatives to the Phosal 53 MCT-based ones. All vehicle formulations contained 20% w/w PEG 400 and 10% w/w Polyoxyl 35 castor oil to emulsify oleic acid. An important formulation variable was the type of antioxidants. Another was the presence or absence of 5% w/w ethanol. The antioxidants were introduced when the first stability study indicated that this formulation was prone to oxidative degradation. The 5% ethanol in carrier formulation F13-13 was introduced to reduce or eliminate phase separation under refrigerated conditions. Dog bioavailability was found to be quite high for all formulations studied (Table 8B). Therefore the drivers for formulation selection at this stage were drug solubility and physical stability.

Except for carrier formulation F13-13 (with ethanol), the drug solubility measured at room temperature ranged from 10.5 to 11.4% w/w, i.e., enough to sustain a drug loading of 7.5% w/w with a 50% solubility margin. Unfortunately, addition of 5% ethanol reduced drug solubility at room temperature to 7.8% w/w. Since phase separation at 5° C. was deemed unacceptable, drug solubility in carrier F13-13 was also measured at 5° C. where it was found to be higher than at room temperature (10.3% w/w). This unusual inverse temperature effect was verified with multiple tests at each temperature (n=3). The relatively low drug solubility in F13-13 also means that the maximum drug loading would have to be reduced to 5% w/w if this formulation were selected for the FIM study.

Effect of Dilution Studies in Apple Juice:

Bioavailability in dog of formulations F13-12 and F13-13 was also studied after a 1:20 w/w dilution in apple juice. The drug loading was 7.5% w/w and dose was 100 mg in both cases. An emulsion formed readily after mixing with apple juice. Based on visual observation only, the emulsion appeared stable for at least 30 minutes. The dilute suspensions used in the dog studies were administered by gavage immediately after mixing. The dog PK results obtained with apple juice were compared against historical data obtained with the same sets of dog fed with soft gel capsules filled with the undiluted formulation. The results are summarized in Table 9.

### Stability

Potency

Stability study #1: F11 vs. F13 with 2.5% w/w API in bottles and syringes

Potencies are calculated from the actual drug substance amount used during manufacturing, corrected for known impurities in lot 1251524-0. The main results of this study can summarized as follows:
Phosal 53 MCT-based formulation F11 was much more stable than oleic acid-based formulation F13, irrespective of container and storage conditions
Potency loss during manufacturing was significant in the case of F13 while it was smaller (bottles) or negligible (syringes) in the case of F11
Stability was higher in HSW syringes than in Baxa syringes
No significant potency loss was detected in HSW syringes after 4 weeks of storage at 25° C./60% RH
The potency loss in F13 after 4 weeks of storage at 25° C./60% RH when stored in syringes ranged from 8 to 13%

That the stability of F11 in bottles was not quite as high as in the best syringe could be surprising at first. However, this anomaly can be explained by the fact that, due to a manufacturing defect, the caps came loose on the amber bottles stored in the humidity chambers, most likely, leading to mass transport in and out of the bottle before the problem was discovered and the caps replaced.

Stability Study #2: Effect of Antioxidants on the Stability of Oleic Acid Based Formulations

The formulations used in the second stability study are described in Table 10. F13 is the control without added antioxidant. Solutions F13-3 through F13-5 were prepared to explore the effect of increasing amounts of added BHT. Solutions F13-6 through F13-9 were prepared to study the combined effect of citric acid and BHT, while solutions F13-10 through F13-1 were prepared to study the combined effect of ascorbic acid and BHT. Both citric acid and ascorbic acid were dissolved in PEG 400 first, i.e., before the PEG 400 was mixed with the other excipients. Except for some controls made in air, all other solutions were manufactured under a nitrogen blanket. Drug loading was 2.5% w/w in all cases.

A summary of the stability of the above 10 formulations as described herein. Potencies are calculated from the actual amount of drug substance used during manufacturing, corrected for known impurities. The main results can summarized as follows:
Potency loss during manufacturing was significant in all cases (6-8%), although less than in the stability study #1 (10%)
Increasing BHT level did not reduce potency loss
Citric acid in combination with BHT did not reduce potency loss
Ascorbic acid in combination with BHT appeared to reduce potency loss during manufacturing by 1-2%
No significant potency loss was detected as a result of storage at 5° C. for 5 weeks
The potency loss after 4 weeks of storage at 25° C./60% RH was as high as 6%
The potency loss after 2 weeks of storage at 40° C./75% RH was as high as 8%

Each of the formulations also was similar by related-substances HPLC, exhibiting two “oxidation” peaks. It was noted, though, that chromatographic baseline showed significant interference from the excipients, perhaps masking important information.

Physical Appearance

Stability Study #2:

The samples used in stability study #2 were also examined for color and phase separation after storage under various conditions for 4 weeks. The results are summarized in Table 10. The main results can be summarized as follows:
After storage at 5° C. for 4 weeks, the original straw color of all formulations was maintained.
After storage at 25° C./60% RH for 4 weeks, all samples turned from straw to pink, with the notable exception of the 2 samples containing ascorbic acid.
After storage at 40° C./75% RH for 4 weeks, all samples turned from straw to pink. The color change in the 2 samples containing ascorbic acid was less pronounced, than at 25° C./60% RH, however.
After storage at 5° C. for 4 weeks, all sample experienced phase separation in the form of a sediment and a surface film. In all cases, this phase separation was thermally reversible upon warming the samples to room temperature.
   Complementary Stability with Additional Formulations:

In an effort to improve upon the results observed with the second stability study, an accelerated qualitative stability study was performed on 3 additional formulations defined in Table 11B. F13-13 had 5% ethanol to eliminate phase separation under refrigerated storage conditions. F13-14 and F13-15 had vitamin E and ascorbyl palmitate as antioxidants. In all 3 cases, the drug loading was 7.5% w/w, as these solutions were excess from a dog study, instead of being part of a formal stability study. The solutions were stored at 5° C. and also subjected to accelerated degradation by exposure to 50° C. overnight. The results presented in Table 11B can be summarized as follows:
After storage at 50° C. overnight, all formulations turned from straw to pink, except for F13-15 with 0.18% ascorbyl palmitate.
After storage at 5° C., the 2 formulations without ethanol all experienced phase separation identical in the form of a sediment and a surface film. In the case of F13-13 (with ethanol), however, sedimentation was eliminated and the surface film was very light and thermally reversible.
   Discussion

Both the Phosal and oleic acid-based formulation efforts have yielded carriers with drug solubility and dog PK (after apple juice dilution). This was achieved by combining the lipids with suitable levels of PEG 400 and emulsifiers. Alcohol was also added to reduce viscosity and to reduce phase separation under refrigerated conditions. The main difference between the two carrier families lies with stability. While F11 showed virtually no potency loss during manufacturing and storage for 4 weeks at 25° C. in HSW syringes, F13 and derivative formulae suffered from both a manufacturing loss and degradation during storage. The addition of antioxidants was not successful at reducing these potency losses in any significant manner. Ascorbic acid and ascorbyl palmitate did slow down the discoloration process from straw to pink significantly, but with little concomitant decrease in potency loss.

Potency Loss:

The mechanisms for potency loss were not elucidated at the time of the project transfer between LC and LU. For more information on this subject, the reader is encouraged to read future memos originating from the LU team.

Turbidity Under Refrigerated Conditions:

Both active and placebo oleic acid-based formulations become turbid when stored under refrigerated conditions (5° C.). This phase separation is due to the presence of saturated fatty acid impurities in the oleic acids, such as palmitic acid, myristic acid and stearic acid. The temperature below which phase separation occurs is referred to as the “titer” in the certificates of analysis. The titer is close to 5° C. Phase separation under refrigerated conditions takes two forms: (1) turbidity which tends to settle to the bottom over time in the absence of vibrations and (2) a thin solid film floating at the surface. Turbidity has been reported earlier. It is easy to see and readily disappears with 5% ethanol. By contrast, the thin film at the surface is more difficult to see and may persist, even with 5% ethanol.

Solubilization Mechanism:

When selected formulations from this study were diluted in aqueous solvents and analyzed with polarized light microscopy, “Maltese Cross” patterns were observed. This suggests that the hydrophobic drug might be trapped within multi-layered liposomal structures. It is hypothesized that these structures delay drug recrystallization, possibly allowing absorption by passive transport between the liposomal structures and the intestinal wall.

TABLE 5B
Phosal 53 MCT: composition and compendial status of
ingredients

| | | | | CEDER | |
|---|---|---|---|---|---|
| Ingredients | Supplier | Compendial status | Function | listed? | Other products |
| Lecithin (60.8%) | Phospholipid GmbH | FCC, lecithin | Preliposomal | Yes | numerous in pharma, |
| | | monograph, | structure, | | cosmetic and food |
| | | | Emulsifier | | |
| MCT oil (28.9%) | Sasol, Germany | PH.Eur, Medium | Carrier, solubilizer | Yes | food and parenteral |
| | | Chain triglycerides | | | nutrition |
| Ethanol (5.1%) | Federal Monopoly | USP, (>99.5%) | Viscosity control | Yes | numerous in pharma, |
| | for ethanol | | | | cosmetic and food |
| Glyceryl stearate (3%) | Goldschmidt | FCC, monograph for | Viscosity control, | Yes | numerous in pharma, |
| | | “mono-and | emulsifier | | cosmetic and food |
| | | diglycerides” | | | |
| Oleic acid (2%) | Ludwig Scheins, | to be determined | Viscosity control, | Yes | numerous in pharma, |
| | Germany | | emulsifier | | cosmetic and food |
| Ascorbyl Palmitate | Roche | FCC, NF, E304 | Antioxidant | Yes | numerous in pharma, |
| (0.02%) | | | | | cosmetic and food |

Note:
Ingredient level are approximate only and expressed in % w/w. All ingredients are from non-animal sources.

TABLE 6B
Drug solubility and dog PK data of Phosal 53 MCT formulations

| | | | | | | | | Fasted Dog PK Study | | | |
|---|---|---|---|---|---|---|---|---|---|---|---|
| | PEG | Polysorbate | | Phosal | Drug | Dose | Solubility % | Cₘₐₓ | Tₘₐₓ | | |
| | 400 | 80 | EtOH | 53 MCT | % w/w | (mg) | w/w (n = 1) | (mcg/ml) | (hr) | F (%) | n |
| Baseline | | | 10 | 90 | 6.5 | 100 | 9.7 | 1.69 (0.17) | 1.4 (0.2) | 37.4 (1.9) | 3 |
| F11 | 10 | | | 90 | 10 | 100 | 13.9 | 1.57 (0.28) | 1.5 (0.3) | 27.4 (3.9) | 3 |
| F11-4 | 10 | | | 90 | 7.5 | 100 | 13.9 | 2.31 (0.11) | 1.3 (0.8) | 46.2 (3.4) | 3 |
| F11-5 | 10 | | 5 | 85 | 7.5 | 100 | 12.3 | 1.67 (0.20) | 1.5 (0.3) | 30.9 (2.3) | 3 |
| F11-6 | 10 | 0.5 | 4.5 | 85 | 7.5 | 100 | 12.2 | 2.90 (0.19) | 1.7 (0.7) | 67.7 (7.8) | 3 |
| F11-7 | | 0.5 | 10 | 89.5 | 5 | 100 | Not measured | 3.56 (0.44) | 1.5 (0.3) | 69.1 (5.7) | 3 |

Note:
Standard error in parenthesis

TABLE 7B
Drug solubility and dog PK data of the baseline formulation,
F11-6 and F11-7: effect of apple juice dilution

| | Drug | | | | | |
|---|---|---|---|---|---|---|
| | loading in | | Fasted Dog PK Study | | | |
| | carrier % | | Cₘₐₓ | Tₘₐₓ | | |
| | w/w | Dose (mg) | (mcg/ml) | (hr) | F (%) | n |
| Baseline | 6.5 | 100 | 1.69 (0.17) | 1.4 (0.2) | 37.4 (1.9) | 3 |
| Baseline, diluted in apple juice | 5.0 | 100 | 2.55 (0.85) | 1.0 (0.3) | 40.2 (15.5) | 3 |
| F11-6 | 7.5 | 100 | 2.90 (0.19) | 1.7 (0.7) | 67.7 (7.8) | 3 |
| F11-6, diluted in apple juice | 5.0 | 100 | 2.36 (0.24) | 1.8 (0.2) | 44.6 (12.3) | 3 |
| F11-7 | 5.0 | 100 | 3.56 (0.44) | 1.5 (0.3) | 69.1 (5.7) | 3 |
| F11-7, diluted in apple juice | 5.0 | 100 | 2.47 (0.28) | 1.7 (0.2) | 45.3 (2.5) | 3 |

TABLE 8B
Drug solubility and dog PK data of oleic acid-based
formulations

| | | | | | | | | | | | Fasted Dog PK Study | | | |
|---|---|---|---|---|---|---|---|---|---|---|---|---|---|---|
| | PEG | Crem | Oleic | | | | Asc | Drug | Dose | API solubility % w/w. | Cₘₐₓ | Tₘₐₓ | | |
| | 400 | EL | Acid | BHT | EtOH | Vit. E | palm | % w/w | (mg) | Room temperature | (mcg/ml) | (hr) | F (%) | n |
| F13 | 20 | 10 | 70.0 | | | | | 7.5 | 75 | 11.4 (n = 1) | 1.92 (0.19) | 1.5 (0.3) | 40.7 (3.3) | 3 |
| F13-2 | 20 | 10 | 69.8 | 0.2 | | | | 7.5 | 100 | 10.4 +/− 1.5 (n = 3) | 3.79 (0.48) | 1.3 (0.2) | 63.7 (4.7) | 3 |
| F13-12 | 20 | 10 | 70.0 | | | | | 7.5 | 100 | 10.7 +/− 3.3 (n = 4) | 2.87 (0.15) | 2.3 (0.8) | 57.7 (3.1) | 3 |
| F13-13 | 20 | 10 | 64.95 | 0.05 | 5 | | | 7.5 | 100 | 7.82 +/− 0.095 (n = 3) | 2.94 (0.43) | 1.5 (0.3) | 56.0 (11.7) | 3 |
| F13-14 | 20 | 10 | 69.90 | | | 0.1 | | 7.5 | 100 | 11.2 (n = 1) | 4.21 (0.26) | 2.0 (0.5) | 76.1 (7.4) | 3 |
| F13-15 | 20 | 10 | 69.82 | | | | 0.18 | 7.5 | 100 | 11.1 (n = 1) | 1.98 (0.90) | 1.7 (0.7) | 39.2 (15.1) | 3 |

Notes:
Standard error in parenthesis. API solubility in F13-13 at 5° C. (% w/w): 10.3 (0.1), n = 3

TABLE 9B
Drug solubility and dog PK data of F13-12 and 13-13: effect of
apple juice dilution

| | Drug | | | | | |
|---|---|---|---|---|---|---|
| | loading in | | Fasted Dog PK Study | | | |
| | carrier % | | Cₘₐₓ | Tₘₐₓ | | |
| | w/w | Dose (mg) | (mcg/ml) | (hr) | F (%) | n |
| F13-12 in SGC | 7.5 | 100 | 2.87 (0.15) | 2.3 (0.8) | 57.7 (3.1) | 3 |
| F13-12 diluted in AJ (1:20 w/w) | 7.5 | 100 | 3.84 (0.71) | 1.3 (0.2) | 68.5 (16.1) | 3 |
| F13-13 in SGC | 7.5 | 100 | 2.94 (0.43) | 1.5 (0.3) | 56.0 (11.7) | 3 |
| F13-13 diluted in AJ (1:20 w/w) | 7.5 | 100 | 2.34 (0.20) | 2.0 (0.5) | 41.7 (4.6) | 3 |

Notes:
Standard error in parenthesis.

TABLE 10B
Second stability study: oleic acid formulations with added
antioxidants. Compositions and visual observations

| | F13 | F13-3 | F13-4 | F13-5 | F13-6 | F13-7 | F13-8 | F13-9 | F13-10 | F13-11 |
|---|---|---|---|---|---|---|---|---|---|---|
| PEG 400 | 20 | 20 | 20 | 20 | 20 | 20 | 20 | 20 | 20 | 20 |
| Cremophor EL | 10 | 10 | 10 | 10 | 10 | 10 | 10 | 10 | 10 | 10 |
| Oleic acid | 70 | 69.98 | 69.95 | 69.85 | 69.93 | 69.9 | 69.88 | 69.85 | 69.93 | 69.9 |
| BHT | | 0.02 | 0.05 | 0.15 | 0.02 | 0.05 | 0.02 | 0.05 | 0.02 | 0.05 |
| Citric acid | | | | | 0.05 | 0.05 | 0.1 | 0.1 | | |
| Ascorbic acid | | | | | | | | | 0.05 | 0.05 |
| Color after 4 w @ 5° C. | Straw | Straw | Straw | Straw | Straw | Straw | Straw | Straw | Straw | Straw |
| Color after 4 w @ 25° C./60% RH | Pink | Pink | Pink | Pink | Pink | Pink | Pink | Pink | Straw | Straw |
| Color after 4 w @ 40° C./75% RH | Pink | Pink | Pink | Pink | Pink | Pink | Pink | Pink | Light | Light |
| | | | | | | | | | pink | pink |
| Phase separation after 4 w @ 5° C. | S + F | S + F | S + F | S + F | S + F | S + F | S + F | S + F | S + F | S + F |

Notes:
S = sediment,
F = surface film
Drug loading = 2.5% w/w. All solutions prepared under a nitrogen blanket.

TABLE 11B
Accelerated stability study with additional oleic acid
formulations. Visual observations

| | F13-13 | F13-14 | F13-15 |
|---|---|---|---|
| PEG 400 | 20 | 20 | 20 |
| Cremophor EL | 10 | 10 | 10 |
| Oleic acid | 64.95 | 69.9 | 69.82 |
| BHT | 0.05 | | |
| Ethanol | 5 | | |
| dl alpha tocopherol | | 0.1 | |
| Ascorbyl palmitate | | | 0.18 |
| Color after 50° C. overnight | Pink | Pink | Straw |
| Phase separation @ 5° C. | Light F | S + F | S + F |

Notes:
S = sediment, F = surface film
Drug loading = 7.5% w/w. All solutions prepared under a nitrogen blanket.

TABLE 12B
Major excipients in the oleic acid-based formulations:
compendial status and maximum daily dose, assuming a daily
drug dose of 250 mg

| | | Daily Dose (mg) | | |
|---|---|---|---|---|
| | | Lutrol E400 | | |
| | | PEG 400 | Cremophor | Mednique 6322 |
| | | EP-USP/NF- | EL | oleic acid |
| | Compendial Status | FCC | EP-USP/NF | EP-USP |
| Assuming 5% w/w drug | Max daily dose (mg) | 950 | 475 | 3325 |
| loading: | | | | |
| Assuming 7.5% w/w drug | Max daily dose (mg) | 616.7 | 308.3 | 2158.3 |
| loading: | | | | |
| | Precedent (mg) | 960.8 (1) | 560 (2) | 3600 (3) |

(1) CEDER, PEG400, SGC
(2) CEDER, Polyoxyl 35 castor oil, SGC
(3) Kaletra: 6 SGCs/day

What is claimed is:

1. A pharmaceutical composition comprising a drug-carrier system that comprises a small-molecule drug of low water solubility in solution in a substantially non-aqueous carrier comprising at least one phospholipid and a pharmaceutically acceptable solubilizing agent; wherein said drug-carrier system, when mixed with an aqueous phase, forms a non-gelling, substantially non-transparent liquid dispersion.

2. The composition of claim 1, wherein the drug-carrier system is liquid.

3. The composition of claim 1, wherein the at least one phospholipid is selected from the group consisting of phosphatidylcholines, phosphatidylserines, phosphatidyl-ethanolamines and mixtures thereof.

4. The composition of claim 1, wherein the at least one phospholipid comprises phosphatidylcholine derived from soy lecithin.

5. The composition of claim 1, wherein the solubilizing agent comprises a glycol and/or a glyceride material.

6. The composition of claim 5, wherein the solubilizing agent comprises a glyceride material selected from the group consisting of medium and long chain mono-, di- and triglycerides and mixtures thereof.

7. The composition of claim 5, wherein the solubilizing agent comprises one or more medium chain triglycerides.

8. The composition of claim 1, wherein the carrier further comprises ethanol.

9. The composition of claim 1, wherein the carrier further comprises a pharmaceutically acceptable surfactant.

10. The composition of claim 1, further comprising a capsule shell, suitable for oral administration, wherein the drug-carrier system is encapsulated.

11. The composition of claim 10, wherein the capsule shell is a hard or soft elastic gelatin capsule shell.

12. The composition of claim 1, wherein the drug has a molecular weight not greater than about 500 g/mol.

13. The composition of claim 1, wherein the drug has a solubility in water of less than about 10 μg/ml.

14. The composition of claim 1, wherein the drug is a protein tyrosine kinase inhibitor.

15. The composition of claim 1, wherein the drug is a compound of formula (I) 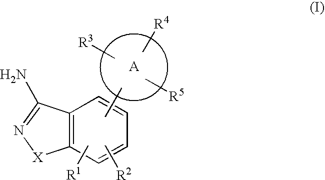
or a therapeutically acceptable salt thereof, where
A is selected from the group consisting of indolyl, phenyl, pyrazinyl, pyridazinyl, pyridinyl, pyrimidyl and thienyl;
X is selected from the group consisting of O, S and NR⁹;
R¹ and R² are independently selected from the group consisting of hydrogen, alkoxy, alkoxyalkoxy, alkoxyalkyl, alkyl, aryl, arylalkyl, aryloxy, aryloxyalkyl, halo, haloalkoxy, haloalkyl, heterocyclyl, heterocyclyl-alkenyl, heterocyclylalkoxy, heterocyclylalkyl, heterocyclyloxyalkyl, hydroxy, hydroxyalkoxy, hydroxyalkyl, (NR^{a}R^{b})alkoxy, (NR^{a}R^{b})alkenyl, (NR^{a}R^{b})alkyl, (NR^{a}R^{b})alkynyl, (NR^{a}R^{b})carbonylalkenyl and (NR^{a}R^{b})-carbonylalkyl;
R³, R⁴ and R⁵ are each independently selected from the group consisting of hydrogen, alkoxy, alkoxyalkoxy, alkyl, halo, haloalkoxy, haloalkyl, hydroxy and LR⁶, provided at least two of R³, R⁴ and R⁵ are other than LR⁶;
L is selected from the group consisting of (CH₂)ₘN(R⁷)C(O)N(R⁸)(CH₂)ₙ and CH₂C(O)NR⁷, where m and n are independently 0 or 1, and wherein each group is drawn with its left end attached to A;
R⁶ is selected from the group consisting of hydrogen, aryl, cycloalkyl, heterocyclyl and 1,3-benzodioxolyl, wherein the 1,3-benzodioxolyl is optionally substituted with one, two or three substituents independently selected from the group consisting of alkenyl, alkoxy, alkoxyalkyl, alkoxycarbonyl, alkyl, alkylcarbonyl, aryl, arylalkoxy, arylalkyl, aryloxy, carboxy, cyano, cycloalkyl, halo, haloalkoxy, haloalkyl, a second heterocyclyl group, heterocyclylalkyl, hydroxy, hydroxyalkyl, nitro, —NR^{c}R^{d} and (NR^{c}R^{d})alkyl;
R⁷ and R⁸ are independently selected from the group consisting of hydrogen and alkyl;
R⁹ is selected from the group consisting of hydrogen, alkenyl, alkoxyalkyl, alkyl, alkylcarbonyl, aryl, heterocyclylalkyl, hydroxyalkyl and (NR^{a}R^{b})alkyl;
R^{a} and R^{b} are independently selected from the group consisting of hydrogen, alkenyl, alkyl, alkylcarbonyl, alkylsulfonyl, aryl, arylalkyl, arylcarbonyl, arylsulfonyl, haloalkylsulfonyl, cycloalkyl, heterocyclyl, heterocyclyl-alkyl and heterocyclylsulfonyl; and
R^{c} and R^{d} are independently selected from the group consisting of hydrogen, alkyl, alkylcarbonyl, aryl, arylalkyl, cycloalkyl and heterocyclyl.

16. The composition of claim 15, wherein the drug is a compound of formula (II) 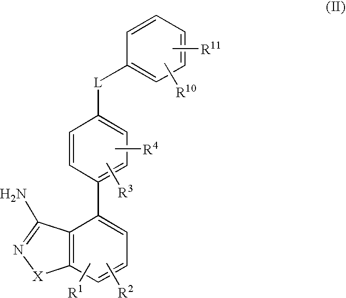
or a therapeutically acceptable salt thereof, where
X is selected from the group consisting of O, S and NR⁹;
R¹ and R² are independently selected from the group consisting of hydrogen, alkoxy, alkoxyalkoxy, alkoxyalkyl, alkyl, aryloxy, aryloxyalkyl, halo, haloalkoxy, haloalkyl, heterocyclyl, heterocyclylalkenyl, heterocyclyl-alkoxy, heterocyclylalkyl, heterocyclyloxyalkyl, hydroxy, hydroxy-alkoxy, hydroxyalkyl, (NR^{a}R^{b})alkoxy, (NR^{a}R^{b})alkenyl, (NR^{a}R^{b})alkyl, (NR^{a}R^{b})carbonylalkenyl and (NR^{a}R^{b})carbonylalkyl;
R³ and R⁴ are independently selected from the group consisting of hydrogen, alkoxy, alkyl, halo, haloalkoxy, haloalkyl and hydroxy;
L is selected from the group consisting of (CH₂)ₘN(R⁷)C(O)N(R⁸)(CH₂)ₙ and CH₂C(O)NR⁷, where m and n are independently 0 or 1, and wherein each group is drawn with its left end attached to the ring substituted with R³ and R⁴;
R⁷ and R⁸ are independently selected from the group consisting of hydrogen and alkyl;
R⁹ is selected from the group consisting of hydrogen, alkenyl, alkoxyalkyl, alkyl, alkylcarbonyl, aryl, heterocyclylalkyl, hydroxyalkyl and (NR^{a}R^{b})alkyl;
R¹⁰ and R¹¹ are independently selected from the group consisting of hydrogen, alkoxy, alkoxyalkyl, alkoxycarbonyl, alkyl, aryloxy, arylalkyl, carboxy, cyano, halo, haloalkoxy, haloalkyl, hydroxy, hydroxyalkyl, nitro and NR^{c}R^{d};
R^{a} and R^{b} are independently selected from the group consisting of hydrogen, alkyl, alkylcarbonyl, alkylsulfonyl, arylsulfonyl, haloalkylsulfonyl and heterocyclylsulfonyl; and
R^{c} and R^{d} are independently selected from the group consisting of hydrogen, alkyl, alkylcarbonyl, aryl, arylalkyl, cycloalkyl, cycloalkylalkyl, heterocyclyl and heterocyclylalkyl.

17. The composition of claim 15, wherein the drug is N-[4-(3-amino-1H-indazol-4-yl)phenyl]-N′-(2-fluoro-5-methylphenyl)urea.

18. The composition of claim 17, comprising per unit dose thereof about 1 to about 500 mg of the drug.

19. The composition of claim 17, comprising per unit dose thereof about 20 to about 200 mg of the drug.

20. The composition of claim 17, wherein the carrier comprises ingredients and amounts thereof selected to provide (a) solubility of the drug of at least about 50 mg/ml at about 25° C.; and (b) a pharmacokinetic profile upon oral administration of the composition in a dog model exhibiting a bioavailability of at least about 25%.

21. The composition of claim 17, wherein the carrier comprises ingredients and amounts thereof selected to provide (a) solubility of the drug of at least about 67 mg/ml at about 25° C.; and (b) a pharmacokinetic profile upon oral administration of the composition in a dog model exhibiting a bioavailability of at least about 30%.

22. The composition of claim 17, wherein the carrier comprises ingredients and amounts thereof selected to provide (a) solubility of the drug of at least about 100 mg/ml at about 25° C.; and (b) a pharmacokinetic profile upon oral administration of the composition in a dog model exhibiting a bioavailability of at least about 50%.

23. The composition of claim 17, wherein, in the carrier, the at least one phospholipid comprises phosphatidylcholine derived from soy lecithin and the solubilizing agent comprises one or more medium chain triglycerides.

24. The composition of claim 23, wherein the carrier comprises about 30% to about 60% phosphatidylcholine, about 25% to about 50% medium chain triglycerides, about 3% to about 15% ethanol, 0% to about 20% of a glycol component and 0% to about 2% of a surfactant component, by weight of the carrier.

25. The composition of claim 23, wherein the carrier comprises Phosal 53 MCT™ or a product substantially equivalent thereto, in an amount of about 50% to 100% by weight of the carrier.

26. The composition of claim 25, wherein the Phosal 53 MCT™ or substantially equivalent product is present in an amount of about 80% to 100% by weight of the carrier.

27. A method of delivering a drug of low water solubility to a subject, the method comprising orally administering a composition of claim 1 that comprises the drug.

28. A pharmaceutical composition comprising a liquid drug-carrier system that comprises a drug in solution in a substantially non-aqueous liquid carrier comprising at least one phospholipid and a pharmaceutically acceptable solubilizing agent; wherein the drug is a compound of formula (I) 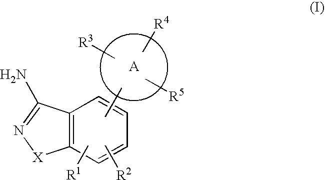
or a therapeutically acceptable salt thereof, where
A is selected from the group consisting of indolyl, phenyl, pyrazinyl, pyridazinyl, pyridinyl, pyrimidyl and thienyl;
X is selected from the group consisting of O, S and NR⁹;
R¹ and R² are independently selected from the group consisting of hydrogen, alkoxy, alkoxyalkoxy, alkoxyalkyl, alkyl, aryl, arylalkyl, aryloxy, aryloxyalkyl, halo, haloalkoxy, haloalkyl, heterocyclyl, heterocyclyl-alkenyl, heterocyclylalkoxy, heterocyclylalkyl, heterocyclyloxyalkyl, hydroxy, hydroxyalkoxy, hydroxyalkyl, (NR^{a}R^{b})alkoxy, (NR^{a}R^{b})alkenyl, (NR^{a}R^{b})alkyl, (NR^{a}R^{b})alkynyl, (NR^{a}R^{b})carbonylalkenyl and (NR^{a}R^{b})-carbonylalkyl;
R³, R⁴ and R⁵ are each independently selected from the group consisting of hydrogen, alkoxy, alkoxyalkoxy, alkyl, halo, haloalkoxy, haloalkyl, hydroxy and LR⁶, provided at least two of R³, R⁴ and R⁵ are other than LR⁶;
L is selected from the group consisting of (CH₂)ₘN(R⁷)C(O)N(R⁸)(CH₂)ₙ and CH₂C(O)NR⁷, where m and n are independently 0 or 1, and wherein each group is drawn with its left end attached to A;
R⁶ is selected from the group consisting of hydrogen, aryl, cycloalkyl, heterocyclyl and 1,3-benzodioxolyl, wherein the 1,3-benzodioxolyl is optionally substituted with one, two or three substituents independently selected from the group consisting of alkenyl, alkoxy, alkoxyalkyl, alkoxycarbonyl, alkyl, alkylcarbonyl, aryl, arylalkoxy, arylalkyl, aryloxy, carboxy, cyano, cycloalkyl, halo, haloalkoxy, haloalkyl, a second heterocyclyl group, heterocyclylalkyl, hydroxy, hydroxyalkyl, nitro, —NR^{c}R^{d}and (NR^{c}R^{d})alkyl;
R⁷ and R⁸ are independently selected from the group consisting of hydrogen and alkyl;
R⁹ is selected from the group consisting of hydrogen, alkenyl, alkoxyalkyl, alkyl, alkylcarbonyl, aryl, heterocyclylalkyl, hydroxyalkyl and (NR^{a}R^{b})alkyl;
R^{a} and R^{b} are independently selected from the group consisting of hydrogen, alkenyl, alkyl, alkylcarbonyl, alkylsulfonyl, aryl, arylalkyl, arylcarbonyl, arylsulfonyl, haloalkylsulfonyl, cycloalkyl, heterocyclyl, heterocyclyl-alkyl and heterocyclylsulfonyl; and
R^{c} and R^{d} are independently selected from the group consisting of hydrogen, alkyl, alkylcarbonyl, aryl, arylalkyl, cycloalkyl and heterocyclyl.

29. The composition of claim 28, wherein the drug is a compound of formula (II) 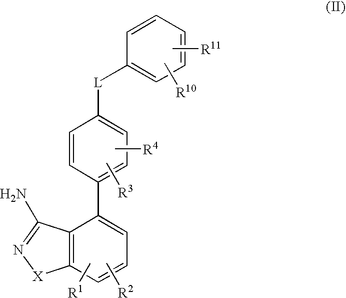
or a therapeutically acceptable salt thereof, where
X is selected from the group consisting of O, S and NR⁹;
R¹ and R² are independently selected from the group consisting of hydrogen, alkoxy, alkoxyalkoxy, alkoxyalkyl, alkyl, aryloxy, aryloxyalkyl, halo, haloalkoxy, haloalkyl, heterocyclyl, heterocyclylalkenyl, heterocyclyl-alkoxy, heterocyclylalkyl, heterocyclyloxyalkyl, hydroxy, hydroxy-alkoxy, hydroxyalkyl, (NR^{a}R^{b})alkoxy, (NR^{a}R^{b})alkenyl, (NR^{a}R^{b})alkyl, (NR^{a}R^{b})carbonylalkenyl and (NR^{a}R^{b})carbonylalkyl;
R³ and R⁴ are independently selected from the group consisting of hydrogen, alkoxy, alkyl, halo, haloalkoxy, haloalkyl and hydroxy;
L is selected from the group consisting of (CH₂)ₘN(R⁷)C(O)N(R⁸)(CH₂)ₙ and CH₂C(O)NR⁷, where m and n are independently 0 or 1, and wherein each group is drawn with its left end attached to the ring substituted with R³ and R⁴;
R⁷ and R⁸ are independently selected from the group consisting of hydrogen and alkyl;
R⁹ is selected from the group consisting of hydrogen, alkenyl, alkoxyalkyl, alkyl, alkylcarbonyl, aryl, heterocyclylalkyl, hydroxyalkyl and (NR^{a}R^{b})alkyl;
R¹⁰ and R¹¹ are independently selected from the group consisting of hydrogen, alkoxy, alkoxyalkyl, alkoxycarbonyl, alkyl, aryloxy, arylalkyl, carboxy, cyano, halo, haloalkoxy, haloalkyl, hydroxy, hydroxyalkyl, nitro and NR^{c}R^{d};
R^{a} and R^{b} are independently selected from the group consisting of hydrogen, alkyl, alkylcarbonyl, alkylsulfonyl, arylsulfonyl, haloalkylsulfonyl and heterocyclylsulfonyl; and
R^{c} and R^{d} are independently selected from the group consisting of hydrogen, alkyl, alkylcarbonyl, aryl, arylalkyl, cycloalkyl, cycloalkylalkyl, heterocyclyl and heterocyclylalkyl.

30. The composition of claim 28, wherein the drug is N-[4-(3-amino-1H-indazol-4-yl)phenyl]-N′-(2-fluoro-5-methylphenyl)urea.

31. A method of treating a condition in a subject for which a protein tyrosine kinase inhibitor is indicated, the method comprising administering to the subject, by a suitable route of administration, a composition of claim 28.

32. The method of claim 31, wherein the route of administration is oral.

33. The method of claim 32, wherein the composition is diluted in a suitable liquid diluent immediately before administering.

34. The method of claim 32, wherein the composition is enclosed in a capsule shell suitable for oral administration.

35. The method of claim 31, wherein the condition is one involving neoplasia.

36. The method of claim 35, wherein the condition involving neoplasia is selected from the group consisting of acute myelogenous leukemia, colorectal cancer, non-small cell lung cancer, hepatocellular carcinoma, non-Hodgkin's lymphoma, ovarian cancer, breast cancer, prostate cancer and kidney cancer.

37. The method of claim 31, wherein the composition comprises N-[4-(3-amino-1H-indazol-4-yl)phenyl]-N′-(2-fluoro-5-methylphenyl)urea as the drug.

38. The method of claim 37, wherein the composition is administered in an amount providing a dose of about 1 mg to about 500 mg of the drug.

39. The method of claim 37, wherein the composition is administered in an amount providing a dose of about 20 mg to about 200 mg of the drug.

40. The composition of claim 1 wherein the drug is a compound of formula (III) 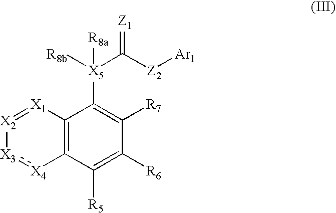
or a pharmaceutically acceptable salt or prodrug thereof, wherein
--- is absent or a single bond;
X₁ is N or CR₁;
X₂ is N or CR₂;
X₃ is N, NR₃, or CR₃;
X₄ is a bond, N, or CR₄;
X₅ is N or C;
provided that at least one of X₁, X₂, X₃, and X₄ is N;
Z₁ is O, NH, or S;
Z₂ is a bond, NH, or O;
Ar₁ is dihydro-1H-indenyl, 1H-indenyl, tetrahydronaphthalenyl, or dihydronaphthalenyl, wherein the Ar₁ group is optionally substituted with 1, 2, 3, 4, or 5 substituents independently selected from alkenyl, alkoxy, alkoxyalkoxy, alkoxyalkyl, alkoxycarbonyl, alkoxycarbonylalkyl, alkyl, alkylcarbonyl, alkylcarbonylalkyl, alkylcarbonyloxy, alkylthio, alkynyl, carboxy, carboxyalkyl, cyano, cyanoalkyl, formyl, formylalkyl, haloalkoxy, haloalkyl, haloalkylthio, halogen, hydroxy, hydroxyalkyl, mercapto, mercaptoalkyl, nitro, (CF₃)₂(HO)C—, —NR_{A}S(O)₂R_{B}, —S(O)₂OR_{A}, —S(O)₂R_{B}, —NZ_{A}Z_{B}, (NZ_{A}Z_{B})alkyl, (NZ_{A}Z_{B})carbonyl, (NZ_{A}Z_{B})carbonylalkyl, or (NZ_{A}Z_{B})sulfonyl, wherein Z_{A} and Z_{B} are each independently hydrogen, alkyl, alkylcarbonyl, formyl, aryl, or arylalkyl;
R₁, R₃, R₅, R₆, and R₇ are each independently hydrogen, alkenyl, alkoxy, alkoxyalkoxy, alkoxyalkyl, alkoxycarbonyl, alkoxycarbonylalkyl, alkyl, alkylcarbonyl, alkylcarbonylalkyl, alkylcarbonyloxy, alkylthio, alkynyl, carboxy, carboxyalkyl, cyano, cyanoalkyl, cycloalkyl, cycloalkylalkyl, formyl, formylalkyl, haloalkoxy, haloalkyl, haloalkylthio, halogen, hydroxy, hydroxyalkyl, mercapto, mercaptoalkyl, nitro, (CF₃)₂(HO)C—, —NR_{A}S(O)₂R_{B}, —S(O)₂OR_{A}, —S(O)₂R_{B}, —NZ_{A}Z_{B}, (NZ_{A}Z_{B})alkyl, (NZ_{A}Z_{B})carbonyl, (NZ_{A}Z_{B})carbonylalkyl or (NZ_{A}Z_{B})sulfonyl; R₂ and R₄ are each independently hydrogen, alkenyl, alkoxy, alkoxyalkoxy, alkoxyalkyl, alkoxycarbonyl, alkoxycarbonylalkyl, alkyl, alkylcarbonyl, alkylcarbonylalkyl, alkylcarbonyloxy, alkylthio, alkynyl, carboxy, carboxyalkyl, cyano, cyanoalkyl, cycloalkyl, cycloalkylalkyl, formyl, formylalkyl, haloalkoxy, haloalkyl, haloalkylthio, halogen, hydroxy, hydroxyalkyl, mercapto, mercaptoalkyl, nitro,(CF₃)₂(HO)C—, —NR_{A}S(O)₂R_{B}, —S(O)₂OR_{A}, —S(O)₂R_{B}, —NZ_{A}Z_{B}, (NZ_{A}Z_{B})alkyl, (NZ_{A}Z_{B})alkylcarbonyl, (NZ_{A}Z_{B})carbonyl, (NZ_{A}Z_{B})carbonylalkyl, (NZ_{A}Z_{B})sulfonyl, (NZ_{A}Z_{B})C(═NH)—, (NZ_{A}Z_{B})C(═NCN)NH—, or (NZ_{A}Z_{B})C(═NH)NH—;
R_{A} is hydrogen or alkyl;
R_{B} is alkyl, aryl, or arylalkyl;
R₈ₐ is hydrogen or alkyl; and
R_{8b} is absent, hydrogen, alkoxy, alkoxycarbonylalkyl, alkyl, alkylcarbonyloxy, alkylsulfonyloxy, halogen, or hydroxy;
provided that R_{8b} is absent when X₅ is N.

41. The composition according to claim 40, wherein the compound is (+)-1-(5-tert-butyl-1-yl)-3-(1H-indazol-4-yl)-urea, or a pharmaceutically acceptable salt or prodrug thereof,

42. A pharmaceutical composition comprising a liquid drug-carrier system that comprises a drug in solution in a substantially non-aqueous liquid carrier comprising at least one phospholipid and a pharmaceutically acceptable solubilizing agent; wherein the drug is a compound of formula (III) 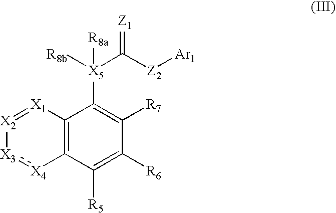
or a pharmaceutically acceptable salt or prodrug thereof, wherein
--- is absent or a single bond;
X₁ is N or CR₁;
X₂ is Nor CR₂;
X₃ is N. NR₃, or CR₃;
X₄ is a bond, N, or CR₄;
X₅ is Nor C;
provided that at least one of X₁, X₂, X₃, and X₄ is N;
Z₁ is O, NH, or S;
Z₂ is a bond, NH, or O;
Ar₁ is dihydro-1H-indenyl, 1H-indenyl, tetrahydronaphthalenyl, or dihydronaphthalenyl, wherein the Ar₁ group is optionally substituted with 1, 2, 3, 4, or 5 substituents independently selected from alkenyl, alkoxy, alkoxyalkoxy, alkoxyalkyl, alkoxycarbonyl, alkoxycarbonylalkyl, alkyl, alkylcarbonyl, alkylcarbonylalkyl, alkylcarbonyloxy, alkylthio, alkynyl, carboxy, carboxyalkyl, cyano, cyanoalkyl, formyl, formylalkyl, haloalkoxy, haloalkyl, haloalkylthio, halogen, hydroxy, hydroxyalkyl, mercapto, mercaptoalkyl, nitro, (CF₃)₂(HO)C—, —NR_{A}S(O)₂R_{B}, —S(O)₂OR_{A}, —S(O)₂R_{B}, —NZ_{A}Z_{B}, (NZ_{A}Z_{B})alkyl, (NZ_{A}Z_{B})carbonyl, (NZ_{A}Z_{B})carbonylalkyl, or (NZ_{A}Z_{B})sulfonyl, wherein Z_{A} and Z_{B} are each independently hydrogen, alkyl, alkylcarbonyl, formyl, aryl, or arylalkyl;
R₁, R₃, R₅, R₆, and R₇ are each independently hydrogen, alkenyl, alkoxy, alkoxyalkoxy, alkoxyalkyl, alkoxycarbonyl, alkoxycarbonylalkyl, alkyl, alkylcarbonyl, alkylcarbonylalkyl, alkylcarbonyloxy, alkylthio, alkynyl, carboxy, carboxyalkyl, cyano, cyanoalkyl, cycloalkyl, cycloalkylalkyl, formyl, formylalkyl, haloalkoxy, haloalkyl, haloalkylthio, halogen, hydroxy, hydroxyalkyl, mercapto, mercaptoalkyl, nitro, (CF₃)₂(HO)C—, —NR_{A}S(O)₂R_{B}, —S(O)₂OR_{A}, —S(O)₂R_{B}, —NZ_{A}Z_{B}, (NZ_{A}Z_{B})alkyl, (NZ_{A}Z_{B})carbonyl, (NZ_{A}Z_{B})carbonylalkyl or (NZ_{A}Z_{B})sulfonyl; R₂ and R₄ are each independently hydrogen, alkenyl, alkoxy, alkoxyalkoxy, alkoxyalkyl, alkoxycarbonyl, alkoxycarbonylalkyl, alkyl, alkylcarbonyl, alkylcarbonylalkyl, alkylcarbonyloxy, alkylthio, alkynyl, carboxy, carboxyalkyl, cyano, cyanoalkyl, cycloalkyl, cycloalkylalkyl, formyl, formylalkyl, haloalkoxy, haloalkyl, haloalkylthio, halogen, hydroxy, hydroxyalkyl, mercapto, mercaptoalkyl, nitro,(CF₃)₂(HO)C—, —NR_{A}S(O)₂R_{B}, —S(O)₂OR_{A}, —S(O)₂R_{B}, —NZ_{A}Z_{B}, (NZ_{A}Z_{B})alkyl, (NZ_{A}Z_{B})alkylcarbonyl, (NZ_{A}Z_{B})carbonyl, (NZ_{A}Z_{B})carbonylalkyl, (NZ_{A}Z_{B})sulfonyl, (NZ_{A}Z_{B})C(═NH)—, (NZ_{A}Z_{B})C(═NCN)NH—, or (NZ_{A}Z_{B})C(═NH)NH—;
R_{A} is hydrogen or alkyl;
R_{B} is alkyl, aryl, or arylalkyl;
R₈ₐ is hydrogen or alkyl; and
R_{8b} is absent, hydrogen, alkoxy, alkoxycarbonylalkyl, alkyl, alkylcarbonyloxy, alkylsulfonyloxy, halogen, or hydroxy;
provided that R_{8b} is absent when X₅ is N.

43. The composition of claim 42 wherein the compound is (+)-1-(5-tert-butyl-1-yl)-3-(1H-indazol-4-yl)-urea, or a therapeutically acceptable salt thereof.

44. The composition of claim 42, comprising per unit dose thereof about 50 to about 900 mg of the drug.

45. The composition of claim 42, wherein the drug-carrier system is liquid.

46. The composition of claim 42, wherein the at least one phospholipid is selected from the group consisting of phosphatidylcholines, phosphatidylserines, phosphatidyl-ethanolamines and mixtures thereof.

47. The composition of claim 42, wherein the at least one phospholipid comprises phosphatidylcholine derived from soy lecithin.

48. The composition of claim 42, wherein the solubilizing agent comprises a glycol and/or a glyceride material.

49. The composition of claim 48, wherein the solubilizing agent comprises a glyceride material selected from the group consisting of medium and long chain mono-, di- and triglycerides and mixtures thereof.

50. The composition of claim 42, wherein the carrier further comprises a pharmaceutically acceptable surfactant.

51. The composition of claim 50, wherein the surfactant is a non-phospholipid surfactant.

52. The composition of claim 51 wherein the surfactant is d-α-tocopheryl polyethylene glycol 1000 succinate (Vitamin E TPGS).

53. The composition of claim 42, wherein the solubilizing agent is not a glycol or a glyceride material.

54. The composition of claim 53, wherein the solubilizing agent is (1,3-bis-(pyrrolidon-1-yl)-butan (VP dimer).

55. The composition of claim 42, further comprising a capsule shell, suitable for oral administration, wherein the drug-carrier system is encapsulated.

56. The composition of claim 55, wherein the capsule shell is a hard or soft elastic gelatin capsule shell.

57. A method of treating a condition in a subject for which a TRPV1 antagonist is indicated, the method comprising administering to the subject, by a suitable route of administration, a composition of claim 42.

58. The method of claim 57, wherein the route of administration is oral.

59. The method of claim 57, wherein the composition is diluted in a suitable liquid diluent immediately before administering.

60. The method of claim 57, wherein the composition is enclosed in a capsule shell suitable for oral administration.

61. The method of claim 57, wherein the condition is selected from the group consisting of pain, neuropathic pain, allodynia, pain associated with inflammation or an inflammatory disease, inflammatory hyperalgesia, bladder overactivity, and urinary incontinence.

62. The method of claim 57, wherein the composition comprises (+)-1-(5-tert-butyl-1-yl)-3-(1H-indazol-4-yl)-urea, or a therapeutically acceptable salt thereof, as the drug.

63. The method of claim 62, wherein the composition is administered in an amount providing a dose of about 1 mg to about 900 mg of the drug.

64. The method of claim 62, wherein the composition is administered in an amount nproviding a dose of about 20 mg to about 200 mg of the drug.
